(19) United States
(12) Patent Application Publication
(10) Pub. No.: US 2025/0074967 A1
(43) Pub. Date: Mar. 6, 2025

(54) IMMUNOMODULATORY FUSION PROTEINS

(71) Applicant: EpicentRx, Inc., La Jolla, CA (US)

(72) Inventors: Christopher LARSON, San Diego, CA (US); Tony R. REID, San Diego, CA (US); Bryan T. ORONSKY, Los Altos Hills, CA (US)

(73) Assignee: EpicentRx, Inc., La Jolla, CA (US)

(21) Appl. No.: 18/772,025

(22) Filed: Jul. 12, 2024

Related U.S. Application Data
(62) Division of application No. 17/150,993, filed on Jan. 15, 2021, now Pat. No. 12,054,530.
Application No. 17/150,993 is a continuation of application No. 15/717,199, filed on Sep. 27, 2017, now Pat. No. 10,906,957.
(60) Provisional application No. 62/484,841, filed on Apr. 12, 2017.
(60) Provisional application No. 62/400,338, filed on Sep. 27, 2016.

(51) Int. Cl. C07K 14/71; A61K 35/768; A61K 38/00; A61K 48/00; A61P 35/00; C07K 16/00; C12N 7/00; C12N 15/86

(52) U.S. Cl. CPC C07K 14/71; A61P 35/00; C07K 16/00; C12N 7/00; C12N 15/86; A61K 35/768; A61K 38/00; A61K 48/00; C07K 2317/522; C07K 2317/524; C07K 2317/53; C07K 2317/76; C07K 2319/01; C07K 2319/30; C12N 2710/10332; C12N 2710/10333; C12N 2710/10343; C12N 2710/10371

(57) ABSTRACT

Provided is a fusion protein, e.g., a cytokine receptor fusion protein, e.g., a TGFβ trap, with a novel linker sequence to permit the fusion protein to functionally optimally, e.g., to permit a cytokine receptor portion of a cytokine receptor fusion protein to bind optimally to its target cytokine. The fusion proteins, or expression vectors encoding for the fusion proteins, e.g., oncolytic adenoviral expression vectors, can be used to treat cell proliferative diseases and disorders, including certain forms of cancer and inflammatory disorders.

### CROSS REFERENCE TO RELATED APPLICATIONS

This application is a continuation of U.S. patent application Ser. No. 15/717,199, filed Sep. 27, 2017, which claims the benefit of, and priority to, U.S. Provisional Application No. 62/400,338, filed Sep. 27, 2016, and U.S. Provisional Application No. 62/484,841 filed Apr. 12, 2017, each of which are hereby incorporated by reference herein in its entirety.

### REFERENCE TO AN ELECTRONIC SEQUENCE LISTING

The content of the electronic sequence listing (203592000810SEQLIST.xml; Size: 155,301 bytes; and Date of Creation: Jun. 25, 2024) is herein incorporated by reference in its entirety.

### FIELD OF THE INVENTION

The field of the invention is molecular biology, specifically immunology and fusion proteins, e.g., cytokine receptor fusion proteins.

### BACKGROUND

Cytokines are small, secreted cell signaling proteins that have a wide range of activities including regulation of cell growth and differentiation and modulation of immune function. Cytokines, cytokine receptors, and certain other immunomodulatory proteins have been used as therapeutics to treat a variety of medical conditions. However, the administration of such proteins, for example, by subcutaneous or vascular routes, can result in inappropriate cellular and extracellular localization, thereby limiting therapeutic activity and/or increasing the risk of toxicity.

Transforming growth factor-β (TGFβ) is a pleiotropic cytokine with immunoregulatory properties, such as the limitation and termination of inflammatory and allergic immune responses (Taylor (2009) J. LEUKOC. BIOL. 85(1):29-33). TGFβ has been implicated in inflammatory, malignant, infectious and autoimmune diseases as well as osteoporosis and fibrosis, including cirrhosis and systemic sclerosis. In particular, persistently high levels of TGFβ in tumors are associated with immune tolerance, angiogenesis, metastasis, and increased tumor extracellular matrix deposition, all of which may drive cancer progression and resistance to therapy.

Several therapeutics have been developed to trap or sequester TGFβ, and, therefore, reduce or modulate TGFβ activity. Examples include monoclonal antibodies directed against TGFβ, for example, fresolimumab, which has been administered in several clinical trials for the treatment of cancer and systemic sclerosis (Connolly et al. (2012) INT. J. BIOL. SCI. 8(7): 964-78).

An alternative approach to monoclonal antibodies includes the use of recombinant Fc-fusion proteins containing a soluble portion of the extracellular domain of the TGFβ type II receptor (TβRII) or the TGFβ type III receptor (TβRIII, or betaglycan) (Connolly et al. (2012) supra). Such molecules, known as TGFβ traps, typically contain extracellular domains of the two chains of the dimeric TGFβ receptor complex. Expression of a soluble TβRII-Fc fusion has been coupled to an oncolytic adenovirus and shown to result in a significant reduction of primary tumor growth and osteolytic bone destruction (Hu et al. (2010) HUM. GENE THER. 21(11): 1623-9).

Despite the efforts to date, there is a need for improved fusion proteins, for example, cytokine receptor fusion proteins, in particular, improved TGFβ receptor fusion proteins that neutralize the biological activity of human TGFβ for treating disorders in human patients mediated by TGFβ.

### SUMMARY OF THE INVENTION

The invention is based, in part, upon the discovery of linker sequences that improve the function of fusion proteins, e.g., cytokine receptor fusion proteins, e.g., TGFβ type II (TβRII) receptor fusion proteins, e.g., TGFβ traps. The linker sequences may permit a ligand binding portion of a fusion protein (e.g., a cytokine receptor) to bind optimally to a ligand (e.g., a cytokine), provide temporal and spatial colocalization of two or more components of a fusion protein (e.g., two subunits of a dimeric cytokine), optimize expression from an expression vector (e.g., a viral vector), reduce immunogenicity, or provide a cleavage site to allow for release of a component of the fusion protein. For example, the linker sequences may provide sufficient flexibility to allow a ligand binding domain of a cytokine receptor to adopt a native conformation in the context of a fusion protein, and minimize the potential immunogenicity of the fusion protein for use as a therapeutic agent.

In one aspect, the invention provides an isolated fusion protein that comprises, for example, in an N- to C-terminal orientation: a first portion of an extracellular domain, transmembrane domain, or intracellular domain of a cytokine, cytokine receptor, or immunomodulatory protein; an amino acid linker; and at least one of, a second portion of an extracellular domain, transmembrane domain, or intracellular domain of a cytokine, cytokine receptor, or immunomodulatory protein; an immunoglobulin (Ig) hinge region; and an immunoglobulin (Ig) Fc domain. In certain embodiments, the linker comprises from about 5 to about 40 amino acid residues.

In another aspect, the invention provides an isolated fusion protein that comprises, in an N- to C-terminal orientation: a soluble portion of an extracellular domain of a cytokine receptor; an amino acid linker; an immunoglobulin (Ig) hinge region; and an immunoglobulin (Ig) Fc domain; wherein the linker comprises from about 5 to about 40 amino acid residues.

In certain embodiments of any of the foregoing fusion proteins, the amino acid linker may comprise, e.g., from about 5 to about 15, from about 5 to about 20, from about 5 to about 30, from about 10 to about 15, from about 10 to about 20, from about 10 to about 30, from about 10 to about 40, from about 15 to about 20, from about 15 to about 30, or from about 15 to about 40 amino acid residues.

In certain embodiments of any of the foregoing fusion proteins, the amino acid linker sequence is derived from an endogenous human protein, e.g., IgG1, IgG2, IgG3, IgG4, IgA1, IgA2, IgD, IgE, IgM, albumin, or casein. In certain embodiments, the amino acid linker comprises a C-terminal portion of an immunoglobulin (Ig) CH1 domain, e.g., an IgG1, IgG2, IgG3, IgG4, IgA1, IgA2, IgD, IgE, or IgM CH1 domain. In certain embodiments, the amino acid linker comprises an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 60, and SEQ ID NO: 61. In certain embodiments, the amino acid linker comprises a C-terminal portion of an IgG1 CH1 domain, e.g., the amino acid linker comprises an amino acid sequence selected from SEQ ID NO: 1, SEQ ID NO: 60, and SEQ ID NO: 61, e.g., the amino acid sequence of SEQ ID NO: 1.

In certain embodiments of any of the foregoing fusion proteins, the amino acid linker comprises a sequence derived from a cytokine, signaling molecule, immunomodulatory protein or peptide, or a biologically active peptide.

In certain embodiments of any of the foregoing fusion proteins, the amino acid linker comprises a cleavage site, e.g., a proteolytic cleavage site, e.g., a proteolytic cleavage site that is cleaved by a protease present in the endoplasmic reticulum or golgi of a eukaryotic cell. In certain embodiments, the proteolytic cleavage site is a furin cleavage site, e.g., a furin cleavage site comprising the sequence RX₁X₂R, wherein X₁ is any amino acid, and X₂ is Lys or Arg, e.g., a furin cleavage site comprising the sequence RAKR (SEQ ID NO: 51). In certain embodiments of any of the foregoing fusion proteins, the amino acid linker is proteolytically stable in a mammal or plant.

In certain embodiments of any of the foregoing fusion proteins, the soluble portion of an extracellular domain of a cytokine receptor is a soluble portion of an extracellular domain of the human TβRII receptor. For example, in certain embodiments, the soluble portion of an extracellular domain of a cytokine receptor comprises the amino acid sequence of SEQ ID NO: 12 or amino acid residues 23-159 of SEQ ID NO: 12.

In certain embodiments of any of the foregoing fusion proteins, the fusion protein comprises one or more of TGF-β, CD80, CD19, CD20, IL-1, IL-3, IL-4, IL-5, IL-6, IL-8, IL-9, IL-12B/p40, IL-23A/p19, IL27A/p28, IL-27B/EBI3, CD154, CD86, CD137, CD137L, IFN-α, IFN-β, BORIS/CTCFL, FGF, ICAM, IL-24, MAGE, NY-ESO-1, angiostatin, endostatin, acetylcholine, interferon-gamma, DKK1/Wnt, p53, thymidine kinase, an anti-PD-1 antibody heavy chain or light chain, and an anti-PD-L1 antibody heavy chain or light chain, or a functional fragment thereof. For example, in certain embodiments, a fusion protein may comprise: CD80 and CD137L; IL-23A/p19 and IL-12B/p40; or IL-27A/p28 and IL-27B/EBI3.

In certain embodiments of any of the foregoing fusion proteins, the Ig hinge region is selected from an IgG1, IgG2, IgG3, IgG4, IgA1, IgA2, IgD, IgE, and IgM hinge region, and the Ig Fc domain, is selected from IgG1, IgG2, IgG3, IgG4, IgA1, IgA2, IgD, IgE, and IgM Fc domain. In certain embodiments, the Ig hinge region and Fc domain together comprise an amino acid sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21. In certain embodiments, the Ig Fc, Ig hinge region, and Ig CH1 domain are derived from a single immunoglobulin.

In certain embodiments of any of the foregoing fusion proteins, the fusion protein comprises an amino acid sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 62, and SEQ ID NO: 63. In certain embodiments, the fusion protein comprises an amino acid sequence selected from SEQ ID NO: 22, SEQ ID NO: 62, and SEQ ID NO: 63. In certain embodiments, the fusion protein comprises the amino acid sequence of SEQ ID NO: 22.

In certain embodiments of any of the foregoing fusion proteins, the fusion protein comprises an amino acid sequence having greater than 80%, 85%, 90%, 95%, 96%, 97%, 98% or 99% sequence identity to a sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 62, and SEQ ID NO: 63.

In another aspect, the invention provides a dimeric cytokine binding protein comprising two of any of the foregoing fusion proteins covalently linked together, wherein each fusion protein comprises an extracellular domain of a cytokine receptor, and wherein the two extracellular domains together define a binding site for a cytokine.

In another aspect, the invention provides a nucleic acid comprising a nucleotide sequence that encodes for any of the foregoing fusion proteins.

In another aspect, the invention provides an expression vector comprising any of the foregoing nucleic acids. The expression vector may be an oncolytic virus, e.g., the virus may selectively replicate in a hyperproliferative cell and/or selectively express the fusion protein in a hyperproliferative cell. In certain embodiments, the oncolytic virus is an oncolytic adenovirus, e.g., an oncolytic type 2 or type 5 adenovirus.

In certain embodiments of any of the foregoing expression vectors, the nucleotide sequence encoding the fusion protein is inserted into an E1b-19K insertion site located between the start site of E1b-19K and the start site of E1b-55K. In certain embodiments, the E1b-19K insertion site is located between the start site of E1b-19K and the stop site of E1b-19K. In certain embodiments, the E1b-19K insertion site comprises a deletion of about 200 nucleotides, e.g., 203 nucleotides adjacent the start site of E1b-19K. In certain embodiments, the E1b-19K insertion site comprises a deletion corresponding to nucleotides 1714-1916 of the Ad5 genome (SEQ ID NO: 52), or, the nucleotide sequence encoding the fusion protein is inserted between nucleotides corresponding to 1714 and 1916 of the Ad5 genome (SEQ ID NO: 5). In certain embodiments, the nucleotide sequence encoding the fusion protein is inserted between CTGACCTC and TCACCAGG, e.g., the adenovirus comprises, in a 5′ to 3′ orientation, CTGACCTC, the nucleotide sequence encoding the fusion protein, and TCACCAGG.

In certain embodiments of any of the foregoing expression vectors, the adenovirus may comprise a deletion of at least one Pea3 binding site, or a functional portion thereof, e.g., the adenovirus may comprise a deletion of nucleotides corresponding to about −300 to about −250 upstream of the initiation site of E1a or a deletion of nucleotides corresponding to −305 to −255 upstream of the initiation site of E1a. In certain embodiments, the adenovirus may comprise a deletion of nucleotides corresponding to 195-244 of the Ad5 genome (SEQ ID NO: 52), and/or the recombinant adenovirus may comprise the sequence GGTGTTTTGG (SEQ ID NO: 55). In certain embodiments, the recombinant oncolytic adenovirus may comprise a deletion of at least one Pea3 binding site, or a functional portion thereof, and not comprise a deletion of an E2F binding site. In certain embodiments, the adenovirus may comprise a deletion of at least one E2F binding site, or a functional portion thereof. In certain embodiments, the adenovirus may comprise a deletion of at least one E2F binding site, or a functional portion thereof, and not comprise a deletion of a Pea3 binding site.

In certain embodiments of any of the foregoing expression vectors, the adenovirus may comprise an E3 deletion. In certain embodiments, the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments, the E3 deletion site is located between the stop site of pVIII and the start site of Fiber. In certain embodiments, the E3 deletion site is located between the stop site of E3-10.5K and the stop site of E3-14.7K. In certain embodiments, the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent to the stop site of E3-10.5K. In certain embodiments, the E3 deletion comprises a deletion of about 1050 nucleotides adjacent to the stop site of E3-10.5K, e.g., the E3 deletion comprises a deletion of 1064 nucleotides adjacent to the stop site of E3-10.5K. In certain embodiments, the E3 deletion comprises a deletion corresponding to the Ad5 dl309 E3 deletion. In certain embodiments, the E3 deletion comprises a deletion corresponding to nucleotides 29773-30836 of the Ad5 genome (SEQ ID NO: 52).

In certain embodiments, the nucleotide sequence encoding the fusion protein is inserted into the E3 deletion, for example, the nucleotide sequence is inserted between CAGTATGA and TAATAAAAAA (SEQ ID NO: 57), e.g., the adenovirus comprises, in a 5′ to 3′ orientation, CAGTATGA, the nucleotide sequence encoding the fusion protein, and TAATAAAAAA (SEQ ID NO: 57).

In certain embodiments, the oncolytic adenovirus comprises a nucleotide sequence encoding a fusion protein inserted into an E1b-19K insertion site, wherein the insertion site is located between the start site of E1b-19K and the start site of E1b-55K, and/or a modified E1a regulatory sequence, wherein at least one Pea3 binding site, or a functional portion thereof, is deleted.

In another aspect, the invention provides a host cell comprising any of the foregoing the expression vectors. In another aspect, the invention provides a method of producing a fusion protein comprising growing a host cell under conditions to express the fusion protein and purifying the fusion protein. In another aspect, the invention provides a method of expressing a fusion protein in a target cell comprising exposing the cell to an effective amount of any of the foregoing expression vectors. In certain embodiments, the fusion protein is cleaved posttranslationally into two polypeptide chains.

In another aspect, any of foregoing fusion proteins or expression vectors can be used, e.g., to reduce cytokine activity in a subject, thereby treating various medical indications that are mediated by a cytokine, for example, TGFβ. In another aspect, any of the foregoing fusion proteins or expression vectors can be used to inhibit proliferation of tumor cells in vitro and/or in vivo, inhibit tumor growth in a subject in need thereof, or treat cancer in a subject in need thereof. The subject may be, e.g., an animal, e.g., a mammal, e.g., a human, e.g., a pediatric human. For example, when administered to a human subject with cancer, the fusion proteins or expression vectors inhibit or reduce tumor growth, or, reduce the tumor load, in the subject.

In certain embodiments, the cancer may be selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, liver cancer, hepatocellular carcinoma, cholangiocarcinoma, brain and central nervous system cancer, thyroid cancer, parathyroid cancer (e.g., parathyroid carcinoma), endometrial cancer, neuroendocrine cancer, lymphoma (e.g., Hodgkin and non-Hodgkin), leukemia, merkel cell carcinoma, gastrointestinal stromal tumors, multiple myeloma, uterine cancer, a sarcoma, kidney cancer, ocular cancer, pancreatic cancer, and a germ cell cancer (e.g., ovarian germ cell cancer). In certain embodiments, the cancer may be selected from leukemia, breast cancer, lung cancer, pancreatic cancer, endometrial cancer, ovarian cancer, prostate cancer, cervical cancer, brain cancer, skin cancer, colorectal cancer, gastric cancer, head and neck cancer, and leukemia.

In certain embodiments, the fusion protein or expression vector is administered in combination with one or more therapies selected from surgery, radiation, chemotherapy, immunotherapy, hormone therapy, and virotherapy. In certain embodiments, the fusion protein or expression vector is administered in combination with a lymphocyte, e.g., a T-cell, e.g., a CAR T-cell.

Any of the foregoing fusion proteins or expression vectors can also be used to treat an inflammatory condition or infection in a subject in need thereof.

These and other aspects and advantages of the invention are illustrated by the following figures, detailed description and claims.

### DESCRIPTION OF THE DRAWINGS

The invention can be more completely understood with reference to the following drawings.

FIG. 1A depicts a schematic of a dimeric cytokine receptor on the cell surface (left), an antibody (middle), and a receptor-Fc fusion that optimally binds a target cytokine (right). FIG. 1B depicts a receptor-Fc fusion, e.g., a cytokine trap, that is sterically constrained from optimal binding to a target cytokine (left), or that adopts an optimal binding configuration (right).

FIG. 2 depicts a sequence alignment of the amino acid sequences of the human IgG1, IgG2, IgG3, IgG4, IgA1, IgA2, IgD, IgE, and IgM CH1 domains (top) and CH2 domains (bottom).

FIG. 3 depicts a Western blot for phosphorylated Smad2 following treatment of reporter cells with TGFB and/or the TGFB type II receptor fusion proteins hTGFBR-IgG-1 and hTGFBR-Fc as indicated. Total Smad2 and Smad3 were used as a loading control. TGFB activity was markedly reduced by hTGFBR-IgG-1 compared to hTGFBR-Fc.

FIG. 4 depicts a Western blot for phosphorylated Smad2 following treatment of reporter cells with TGFβ and/or the TGFβ type II receptor fusion proteins hTGFβR-IgG1-1 (1), hTGFβR-IgG1-2 (2), hTGFβR-IgG1-3 (3), and hTGFβR-IgG1-4 (4) as indicated. B-actin was used as a loading control.

FIGS. 5A-5C depict tumor volumes in mice following treatment with the indicated virus. FIG. 5A shows the buffer control. FIG. 5B shows the Adenoviral vector control. FIG. 5C shows the Adenoviral vector expressing the hTGFβR-IgG1 construct. Each line represents the tumor volume of one mouse.

FIGS. 6A-6B depict Western blots for phosphorylated Smad2 following treatment of the indicated cell lines with TGFβ and/or the indicated virus. Total Smad2 and Smad3 were used as a loading control. FIG. 6A compares the buffer control, the Adenoviral vector control, the Adenoviral vector expressing the mTGFβR-IgG1 construct, and the Adenoviral vector expressing the hTGFβR-IgG1 construct. FIG. 6B compares just the Adenoviral vector control and the Adenoviral vector expressing the hTGFβR-IgG1 construct.

### DETAILED DESCRIPTION

The invention provides an isolated fusion protein for use in the treatment of a variety of medical conditions, for example, in inhibiting proliferation of a tumor cell, inhibiting tumor growth, treating cancer, treating an inflammatory condition, or treating an infection, in a subject. Exemplary fusion proteins comprise: a first portion of an extracellular domain, transmembrane domain, or intracellular domain of a cytokine, cytokine receptor, or immunomodulatory protein; an amino acid linker; and at least one of, a second portion of an extracellular domain, transmembrane domain, or intracellular domain of a cytokine, cytokine receptor, or immunomodulatory protein; an immunoglobulin (Ig) hinge region; and an immunoglobulin (Ig) Fc domain. In certain embodiments, the linker comprises from about 5 to about 40 amino acid residues. Exemplary fusion proteins of the invention include cytokine traps.

A cytokine trap, e.g. a TGFβ trap, is a molecule containing a soluble portion of the extracellular domain of a cytokine receptor, e.g., a TGFβ receptor, e.g., the TGFβ type II receptor (TβRII), designed to bind or otherwise sequester a target cytokine. In a cytokine trap, the extracellular domain of a cytokine receptor may be fused to an immunoglobulin (Ig) hinge region and immunoglobulin (Ig) Fc domain which can allow, e.g., for increased stability, Fc effector functions and/or multimerization, e.g., dimerization. Dimerization afforded by fusion to an Ig hinge region and Ig Fc domain is particularly advantageous for cytokine receptors that exist as dimeric receptor complexes on the cellular surface, such as, e.g., TβRII.

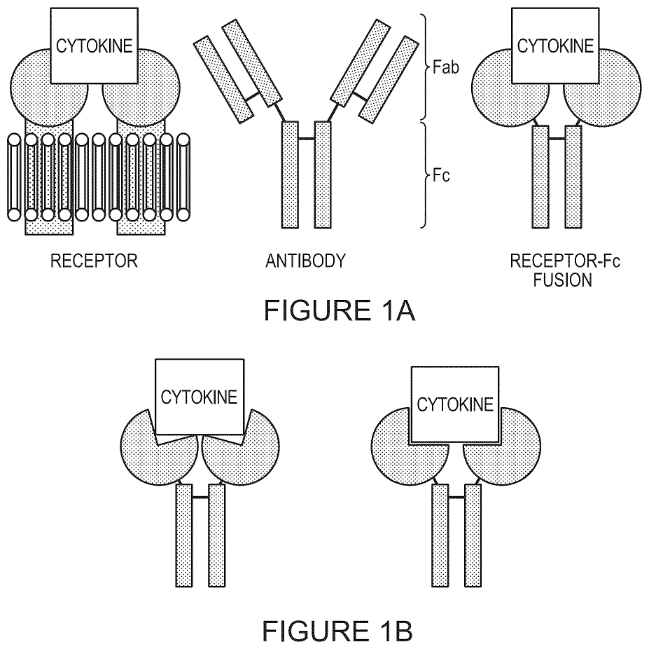

Conventional cytokine traps, e.g., TGFβ traps, comprise two polypeptide chains, each polypeptide chain comprising a soluble portion of an extracellular domain of a cytokine receptor fused to an Ig hinge region and an Ig Fc domain. The soluble portion of the extracellular domain of the cytokine receptor typically is fused directly to the Ig hinge region, without any intervening sequence. The two polypeptide chains are covalently linked by disulfide bonds between cysteine residues in each of the Ig hinge regions. Each polypeptide chain provides a soluble portion of an extracellular domain of a cytokine receptor, e.g., TβRII, and the two soluble portions of an extracellular domain of a cytokine receptor together define a binding site for a cytokine. A schematic representation of a dimeric cytokine receptor, an immunoglobulin (antibody) molecule, and a dimeric protein comprising two covalently linked fusion proteins each comprising a soluble portion of an extracellular domain of a cytokine receptor fused to an Ig hinge region and an Ig Fc domain is depicted in FIG. 1A.

The invention is based, in part, upon the discovery that conventional cytokine traps comprising a fusion protein of a soluble portion of an extracellular domain of a cytokine receptor to an Ig hinge region and Ig Fc domain, e.g. TGFβ traps, do not optimally bind their target cytokine. For example, a conventional TGFβ trap does not provide sufficient flexibility between the two TβRII ligand binding domains to allow the two TβRII ligand binding domains to come together in an optimal configuration to define a TGFβ binding site.

Thus, in one aspect, the invention provides an isolated fusion protein that comprises, in an N- to C-terminal orientation: a soluble portion of an extracellular domain of a cytokine receptor; an amino acid linker; an immunoglobulin (Ig) hinge region; and an immunoglobulin (Ig) Fc domain; wherein the linker comprises from about 5 to about 40 amino acid residues. The linker sequence allows, e.g., the binding domain in the extracellular domain of the cytokine receptor to bind optimally to its target cytokine. This is especially important when the cytokine binding protein is a dimer that comprises two of the foregoing fusion proteins that together define a binding site to bind the target cytokine. Without the linker, the two binding domains may be sterically constrained from forming the optimal binding site (FIG. 1B). Various features and aspects of the invention are discussed in more detail below.

### I. Fusion Proteins

Exemplary fusion proteins may comprise: a first portion of an extracellular domain, transmembrane domain, or intracellular domain of a cytokine, cytokine receptor, or immunomodulatory protein; an amino acid linker; and at least one of, a second portion of an extracellular domain, transmembrane domain, or intracellular domain of a cytokine, cytokine receptor, or immunomodulatory protein; an immunoglobulin (Ig) hinge region; and an immunoglobulin (Ig) Fc domain. It is contemplated that the first portion of an extracellular domain, transmembrane domain, or intracellular domain of a cytokine, cytokine receptor, or immunomodulatory protein may be the same or different from the second portion of an extracellular domain, transmembrane domain, or intracellular domain of a cytokine, cytokine receptor, or immunomodulatory protein

For example, a disclosed fusion protein may comprise, in an N- to C-terminal orientation: a soluble portion of an extracellular domain of a cytokine receptor; an amino acid linker; an immunoglobulin (Ig) hinge region; and an immunoglobulin (Ig) Fc domain; wherein the linker comprises from about 5 to about 40 amino acid residues.

Exemplary cytokines include IL-1α, IL-1β, IL-18, IL-4, IL-9, IL-13, IL-3, IL-5, IL-6, IL-11, G-CSF, LIF, OSM, IL-10, IL-20, IL-14, IL-16, IL-17, IFN-α, IFN-β, IFN-γ, CD154, LT-β, TNF-β, 4-1BBL APRIL, CD153, CD178, LIGHT, TALL-1, TRAIL, TWEAK, TRANCE, TGF-β1, TGF-β2, TGF-β3, Epo, Tpo, Flt-3L, SCF, M-CSF, and MSP.

As used herein, an “immunomodulatory” protein refers to a protein that modulates the function of the immune system of a subject. Immunomodulatory proteins may, for example, modulate the function of, e.g., B-cells, T cells and/or the production of antibodies. Exemplary immunomodulatory proteins include checkpoint inhibitors. Exemplary immunomodulatory proteins may include, e.g., PD-1, or PD-L1, or any protein that modulates the activity thereof. Further exemplary immunomodulatory proteins may include an anti PD-1 antibody or anti-PD-L1 antibody.

As used herein, a “soluble portion of an extracellular domain of a cytokine receptor” refers to any extracellular domain of a cytokine receptor or fragment of an extracellular domain of a cytokine receptor that is capable of binding to a target cytokine. It is understood that the soluble portion of an extracellular domain of a cytokine receptor also contemplates portions of the extracellular domain that comprise a binding domain that, either alone or in combination with a second binding domain (e.g., in the case of dimeric fusion proteins) is capable of binding to a target cytokine.

Exemplary cytokine receptors include type I cytokine receptors (e.g., GM-CSF receptors, G-CSF receptors, type I IL receptors, Epo receptors, LIF receptors, CNTF receptors, or TPO receptors), type II cytokine receptors (e.g, IFN-alpha receptors (e.g., IFNAR1 or IFNAR2), IFN-beta receptors, IFN-gamma receptors (e.g., IFNGR1 or IFNGR2), chemokine receptors (e.g., CC chemokine receptors, CXC chemokine receptors, CX3C chemokine receptors, or XC chemokine receptors), tumor necrosis factor superfamily receptors (TNFRs; e.g., TNFRSF5/CD40, TNFRSF8/CD30, TNFRSF7/CD27, TNFRSF1A/TNFR1/CD120a, or TNFRSF1B/TNFR2/CD120b), TGFβ superfamily receptors (e.g., TGFβ type I receptor or TGFβ type II receptor), or immunoglobulin (Ig) superfamily receptors (e.g., interleukin-1 receptors, CSF-1R, PDGFR (e.g., PDGFRA or PDGFRB), or SCFR). Preferred cytokine receptors include dimeric cytokine receptors, e.g., TGFβ superfamily receptors, e.g., the human TGFβ type II receptor (TβRII). In certain embodiments, the soluble portion of an extracellular domain of a cytokine receptor is a soluble portion of an extracellular domain of the human TGFβ type II receptor (TβRII), e.g., comprising the amino acid sequence of SEQ ID NO: 12, or an amino acid sequence having greater than 85%, 90%, 95%, 96%, 97%, 98% or 99% sequence identity to SEQ ID NO: 12, and/or a fragment thereof that comprises a binding domain that binds to TGFβ.

The soluble portion of the extracellular domain of a cytokine receptor retains its ability to bind its native ligand. In certain embodiments, the soluble portion of the extracellular domain retains at least 50%, 60%, 70%, 80%, 90%, or 95% of the binding activity to its native ligand when compared to the full length cytokine receptor.

In certain embodiments, the fusion protein can comprise, e.g., one or more of TβRII, TGF-β, CD80, CD19, CD20, IL-1, IL-3, IL-4, IL-5, IL-6, IL-8, IL-9, IL-12B/p40, IL-23A/p19, IL-27A/p28, IL-27B/EBI3, CD154, CD86, CD137, CD137L, IFN-α, IFN-β, BORIS/CTCFL, FGF, ICAM, IL-24, MAGE, NY-ESO-1, angiostatin, endostatin, acetylcholine, interferon-gamma, DKK1/Wnt, p53, thymidine kinase, an anti-PD-1 antibody heavy chain or light chain, and an anti-PD-L1 antibody heavy chain or light chain, or a functional fragment thereof. For example, a fusion protein may comprise: CD80 and CD137L; IL-23A/p19 and IL-12B/p40; or IL-27A/p28 and IL-27B/EBI3.

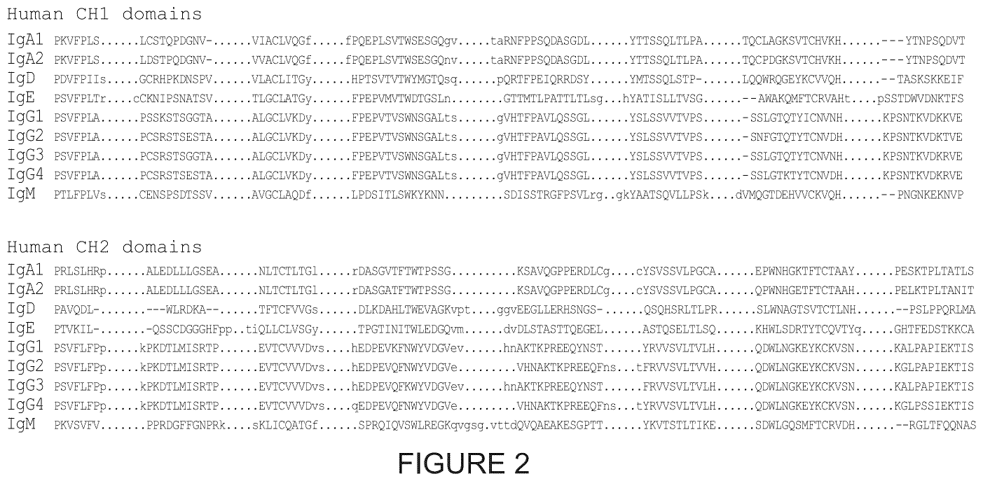

As used herein, the term “immunoglobulin (Ig) hinge region” refers to the amino acid sequence that typically connects CH1 and CH2 domains of an immunoglobulin heavy chain constant region. An Ig hinge region may include, e.g., one or more cysteine residues capable of forming disulfide bonds with cysteine residues in another protein chain. As used herein, the term “immunoglobulin (Ig) Fc domain” refers to a fragment of an immunoglobulin heavy chain constant region that is capable of binding to an Fc receptor. An Ig Fc domain may include, e.g., an immunoglobulin (Ig) CH2 and CH3 domain. Boundaries between Ig CH1, CH2, and CH3 domains are well known in the art, and can be found, e.g., in the PROSITE database (available on the world wide web at prosite.expasy.org). For clarity, alignments of the amino acid sequences of the human IgG1, IgG2, IgG3, IgG4, IgA1, IgA2, IgD, IgE, and IgM CH1 and CH2 domains are provided in FIG. 2.

In certain embodiments, the Ig hinge region is selected from an IgG1, IgG2, IgG3, IgG4, IgA1, IgA2, IgD, IgE, and IgM hinge region, and the Ig Fc domain, is selected from an IgG1, IgG2, IgG3, IgG4, IgA1, IgA2, IgD, IgE, and IgM Fc domain. In certain embodiments, the Ig hinge region and Fc domain together comprise an amino acid sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21. In certain embodiments, the Ig hinge region and Fc domain together comprise an amino acid sequence having greater than 85%, 90%, 95%, 96%, 97%, 98% or 99% identity with a sequence selected from SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21.

The amino acid linker may permit a ligand binding portion of a fusion protein (e.g., a cytokine receptor) to bind optimally to a ligand (e.g., a cytokine), provide temporal and spatial colocalization of two or more components of a fusion protein (e.g., two subunits of a dimeric cytokine), optimize expression from an expression vector (e.g., a viral vector), reduce immunogenicity, or provide a cleavage site to allow for release of a component of the fusion protein.

The amino acid linker may comprise, e.g., from about 5 to about 15, from about 5 to about 20, from about 5 to about 25, from about 5 to about 30, from about 5 to about 35, from about 5 to about 40, from about 10 to about 15, from about 10 to about 20, from about 10 to about 25, from about 10 to about 30, from about 10 to about 35, from about 10 to about 40, from about 15 to about 20, from about 15 to about 25, from about 15 to about 30, from about 15 to about 35, or from about 15 to about 40 amino acid residues. The amino acids in the linker can be naturally occurring amino acids or modified amino acids.

In certain embodiments, the amino acid linker sequence is derived from an endogenous human protein, e.g., IgG1, IgG2, IgG3, IgG4, IgA1, IgA2, IgD, IgE, IgM, albumin, or casein. In certain embodiments, the amino acid linker comprises a C-terminal portion, for example, from about 5 to about 40 amino acids, of an immunoglobulin (Ig) CH1 domain, e.g., an IgG1, IgG2, IgG3, IgG4, IgA1, IgA2, IgD, IgE, or IgM CH1 domain. In certain embodiments, the amino acid linker comprises an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9. SEQ ID NO: 10, SEQ ID NO: 11, SEQ ID NO: 60, and SEQ ID NO: 61. In certain embodiments, the amino acid linker comprises a sequence having greater than 80%, 85%, 90%, 95%, 96%, 97%, 98% or 99% sequence identity to an amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9. SEQ ID NO: 10, SEQ ID NO: 11, SEQ ID NO: 60, and SEQ ID NO: 61.

A protein or polypeptide is “derived from” a reference protein or polypeptide if it comprises an amino acid sequence that is substantially similar to all or a corresponding portion of the wild-type amino acid sequence of the reference protein or polypeptide. In certain embodiments, a protein or polypeptide that is derived from a wild-type protein or polypeptide may have one or more amino acid substitutions relative to the wild-type protein or polypeptide. For example, it is contemplated that a protein or polypeptide that is derived from a wild-type protein or polypeptide may have greater than 70%, 75%, 80%, 85%, 90%, 95%, 96%, 97%, 98% or 99% sequence identity to the wild-type protein or polypeptide. Further, it is contemplated that a protein or polypeptide that is derived from a wild-type protein or polypeptide may contain on more conservative substitutions relative to the wild-type protein or polypeptide. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

In certain embodiments, the amino acid linker sequence is derived from a cytokine, signaling molecule, immunomodulatory protein or peptide, or a biologically active peptide.

Further contemplated linker sequences include glycine- and serine-rich linkers, e.g., (G₄S)₃ (SEQ ID NO: 49). Additional exemplary linker sequences are disclosed, e.g., in George et al. (2003) PROTEIN ENGINEERING 15:871-879 and U.S. Pat. Nos. 5,482,858 and 5,525,491.

In certain embodiments, the amino acid linker may comprise a cleavage site, e.g., a proteolytic or a non-proteolytic cleavage site. In certain embodiments, the proteolytic cleavage site is cleaved by a protease present in a specific tissue, organelle or intracellular compartment. In certain embodiments, the linker comprises a proteolytic cleavage site and two cysteine residues that result in a disulfide linkage following proteolytic cleavage. In certain embodiments, the proteolytic cleavage site is cleaved by a protease selected from a matrix metalloproteinase (MMP), furin, PC1, PC2, PC3, cathepsin B, proteinase 3, and caspase 3. In certain embodiments, the cleavage site is a proteolytic cleavage site that is cleaved by a protease that is present in the endoplasmic reticulum or golgi of a eukaryotic cell. In certain embodiments, the proteolytic cleavage site is a furin cleavage site. Furin is a protease that is ubiquitously expressed and is localized to the golgi, where it recognizes the consensus sequence RX₁X₂R, wherein X₁ is any amino acid, and X₂ is Lys or Arg, and cleaves after the final Arg. Furin plays a biological role in cleaving propeptides of proteins that are trafficked through the golgi. Accordingly, in certain embodiments the proteolytic cleavage site is a furin cleavage site comprising the sequence RX₁X₂R, wherein X₁ is any amino acid, and X₂ is Lys or Arg, e.g., a furin cleavage site comprising the sequence RAKR (SEQ ID NO: 51).

In certain embodiments, the Ig Fc, Ig hinge region, and Ig CH1 domain are derived from a single immunoglobulin.

In certain embodiments, the fusion protein comprises an amino acid sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 62, and SEQ ID NO: 63. In certain embodiments, a disclosed fusion protein comprises an amino acid sequence having greater than 80%, 85%, 90%, 95%, 96%, 97%, 98% or 99% sequence identity to a sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 62, and SEQ ID NO: 63.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent

Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In one aspect the invention provides a cytokine binding protein comprising two fusion proteins, wherein each fusion protein comprises in an N- to C-terminal orientation: a soluble portion of an extracellular domain of a cytokine receptor; an amino acid linker; an immunoglobulin (Ig) hinge region; and an immunoglobulin (Ig) Fc domain; wherein the linker comprises from about 5 to about 40 amino acid residues, wherein the two fusion proteins are covalently linked together, and wherein the two extracellular domains together define a binding site for a cytokine.

The cytokine binding protein may comprise two of the foregoing fusion proteins covalently linked together, wherein each fusion protein comprises an extracellular domain of a cytokine receptor, and wherein the two extracellular domains together define a binding site for a cytokine. The fusion proteins may be covalently linked, e.g., by disulfide bonds between cysteine residues in the Ig hinge region of each fusion protein. In certain embodiments, the fusion proteins, either monomeric or multimeric (e.g., dimeric) retain at least 50%, 60%, 70%, 80%, 90%, or 95% of the binding activity of the target ligand when compared to the native, full length cytokine receptor.

In certain embodiments, a cytokine binding protein of the invention binds a cytokine with a KD of 200 nM, 100 nM, 20 nM, 15 nM, 10 nM, 9 nM, 8 nM, 7 nM, 6 nM, 5 nM, 4 nM, 3 nM, 2 nM, 1 nM, 50 pM, 25 pM or lower. In certain embodiments, a cytokine binding protein of the invention binds a cytokine with a KD of from 200 nM to 100 nM, from 200 nM to 20 nM, from 200 nM to 10 nM, from 200 nM to 5 nM, from 200 nM to 1 nM, from 200 nM to 50 pM, from 200 nM to 25 pM, from 100 nM to 20 nM, from 100 nM to 10 nM, from 100 nM to 5 nM, from 100 nM to 1 nM, from 100 nM to 50 pM, from 100 nM to 25 pM, from 20 nM to 10 nM, from 20 nM to 5 nM, from 20 nM to 1 nM, from 20 nM to 50 pM, from 20 nM to 25 pM, from 10 nM to 5 nM, from 10 nM to 1 nM, from 10 nM to 50 pM, from 10 nM to 25 pM, from 5 nM to 1 nM, from 5 nM to 50 pM, from 5 nM to 25 pM, from 1 nM to 50 pM, from 1 nM to 25 pM, or from 50 pM to 25 pM. In certain embodiments, a cytokine binding protein of the invention binds TGFβ with a K_{D} of 200 nM, 100 nM, 20 nM, 15 nM, 10 nM, 9 nM, 8 nM, 7 nM, 6 nM, 5 nM, 4 nM, 3 nM, 2 nM, 1 nM, 50 pM, 25 pM or lower. In certain embodiments, a cytokine binding protein of the invention binds TGFβ with a K_{D} of from 200 nM to 100 nM, from 200 nM to 20 nM, from 200 nM to 10 nM, from 200 nM to 5 nM, from 200 nM to 1 nM, from 200 nM to 50 pM, from 200 nM to 25 pM, from 100 nM to 20 nM, from 100 nM to 10 nM, from 100 nM to 5 nM, from 100 nM to 1 nM, from 100 nM to 50 pM, from 100 nM to 25 pM, 20 nM to 10 nM, from 20 nM to 5 nM, from 20 nM to 1 nM, from 20 nM to 50 pM, from 20 nM to 25 pM, from 10 nM to 5 nM, from 10 nM to 1 nM, from 10 nM to 50 pM, from 10 nM to 25 pM, from 5 nM to 1 nM, from 5 nM to 50 pM, from 5 nM to 25 pM, from 1 nM to 50 pM, from 1 nM to 25 pM, or from 50 pM to 25 pM. K_{D} values may be determined by methods well known in the art, including surface plasmon resonance or bio-layer interferometry methods.

Exemplary fusion proteins of the invention include proteins comprising an amino acid sequence selected from SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 62, and SEQ ID NO: 63. For clarity, the sequences of the individual elements of these proteins, and the proteins from which the sequences of the individual elements were derived, including the soluble portion of an extracellular domain of a cytokine receptor, the amino acid linker, the Ig hinge region, and the Ig Fc domain, are set forth in TABLE 1.

### TABLE 1

TABLE 1

| | Receptor | Linker | Ig Hinge/ |
|---|---|---|---|
| | Source | Source | Ig Fc Source |
| | Receptor | Linker | Ig Hinge/ |
| Protein | SEQ ID | SEQ ID | Ig Fc SEQ ID |
| SEQ ID | TGFβIIR | IgG1 CH1 | IgG1 |
| NO: 22 | SEQ ID | domain | SEQ ID |
| | NO: 12 | SEQ ID | NO: 13 |
| | | NO: 1 | |
| SEQ ID | TGFβIIR | IgG1 CH1 | IgG1 |
| NO: 62 | SEQ ID | domain | SEQ ID |
| | NO: 12 | SEQ ID | NO: 13 |
| | | NO: 60 | |
| SEQ ID | TGFβIIR | IgG1 CH1 | IgG1 |
| NO: 63 | SEQ ID | domain | SEQ ID |
| | NO: 12 | SEQ ID | NO: 13 |
| | | NO: 61 | |
| SEQ ID | TGFβIIR | IgG2 CH1 | IgG2 |
| NO: 23 | SEQ ID | domain | SEQ ID |
| | NO: 12 | SEQ ID | NO: 14 |
| | | NO: 2 | |
| SEQ ID | TGFβIIR | IgG3 CH1 | IgG3 |
| NO: 24 | SEQ ID | domain | SEQ ID |
| | NO: 12 | SEQ ID | NO: 15 |
| | | NO: 3 | |
| SEQ ID | TGFβIIR | IgG4 CH1 | IgG4 |
| NO: 25 | SEQ ID | domain | SEQ ID |
| | NO: 12 | SEQ ID | NO: 16 |
| | | NO: 4 | |
| SEQ ID | TGFβIIR | IgA1 CH1 | IgA1 |
| NO: 26 | SEQ ID | domain | SEQ ID |
| | NO: 12 | SEQ ID | NO: 17 |
| | | NO: 5 | |
| SEQ ID | TGFβIIR | IgA2 CH1 | IgA2 |
| NO: 27 | SEQ ID | domain | SEQ ID |
| | NO: 12 | SEQ ID | NO: 18 |
| | | NO: 6 | |
| SEQ ID | TGFβIIR | IgD CH1 | IgD |
| NO: 28 | SEQ ID | domain | SEQ ID |
| | NO: 12 | SEQ ID | NO: 19 |
| | | NO: 7 | |
| SEQ ID | TGFβIIR | IgE CH1 | IgE |
| NO: 29 | SEQ ID | domain | SEQ ID |
| | NO: 12 | SEQ ID | NO: 20 |
| | | NO: 8 | |
| SEQ ID | TGFβIIR | IgM CH1 | IgM |
| NO: 30 | SEQ ID | domain | SEQ ID |
| | NO: 12 | SEQ ID | NO: 21 |
| | | NO: 9 | |
| SEQ ID | TGFβIIR | Albumin | IgG1 |
| NO: 31 | SEQ ID | SEQ ID | SEQ ID |
| | NO: 12 | NO: 10 | NO: 13 |
| SEQ ID | TGFβIIR | Casein | IgG1 |
| NO: 32 | SEQ ID | SEQ ID | SEQ ID |
| | NO: 12 | NO: 11 | NO: 13 |
| SEQ ID | mTGFβIIR | mIgG1 | mIgG1 |
| NO: 33 | SEQ ID | CH1 domain | SEQ ID |
| | NO: 34 | SEQ ID | NO: 36 |
| | | NO: 35 | |

### TABLE 2

TABLE 2

| Protein Sequence | Nucleic Acid Sequence |
|---|---|
| SEQ ID NO: 22 | SEQ ID NO: 37 |
| SEQ ID NO: 23 | SEQ ID NO: 38 |
| SEQ ID NO: 24 | SEQ ID NO: 39 |
| SEQ ID NO: 25 | SEQ ID NO: 40 |
| SEQ ID NO: 26 | SEQ ID NO: 41 |
| SEQ ID NO: 27 | SEQ ID NO: 42 |
| SEQ ID NO: 28 | SEQ ID NO: 43 |
| SEQ ID NO: 29 | SEQ ID NO: 44 |
| SEQ ID NO: 30 | SEQ ID NO: 45 |
| SEQ ID NO: 31 | SEQ ID NO: 46 |
| SEQ ID NO: 32 | SEQ ID NO: 47 |

### II. Fusion Protein Production

Methods for producing fusion proteins of the invention are known in the art. For example, DNA molecules encoding a disclosed fusion protein can be chemically synthesized using the sequence information provided herein. Synthetic DNA molecules can be ligated to other appropriate nucleotide sequences, including, e.g., expression control sequences, to produce conventional gene expression constructs encoding the desired fusion protein. Production of defined gene constructs is within routine skill in the art. Exemplary nucleic acid sequences SEQ ID NOs: 37-47, which encode the fusion proteins of SEQ ID NOs: 22-32, can be found in TABLE 2.

Nucleic acids encoding desired fusion proteins can be incorporated (ligated) into expression vectors, which can be introduced into host cells through conventional transfection or transformation techniques. Exemplary host cells are E. coli cells, Chinese hamster ovary (CHO) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the desired fusion protein.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon, and, optionally, may contain enhancers, and various introns. The gene construct can be introduced into eukaryotic host cells using conventional techniques.

A polypeptide comprising a disclosed fusion protein can be produced by growing (culturing) a host cell transfected with an expression vector encoding such protein, under conditions that permit expression of the polypeptide. Following expression, the polypeptide can be harvested and purified or isolated using techniques known in the art, e.g., affinity tags such as Protein A, Protein G, glutathione-S-transferase (GST) and histidine tags.

### III. Viral Vectors

In certain embodiments, a disclosed expression vector is a viral vector. The terms “viral vector” and “virus” are used interchangeably herein to refer to any of the obligate intracellular parasites having no protein-synthesizing or energy-generating mechanism. The viral genome may be RNA or DNA. The viruses useful in the practice of the present invention include recombinantly modified enveloped or non-enveloped DNA and RNA viruses, preferably selected from baculoviridiae, parvoviridiae, picornoviridiae, herpesviridiae, poxyiridae, or adenoviridiae. The viruses may be modified by recombinant DNA techniques to include expression of exogenous transgenes and may be engineered to be replication deficient, conditionally replicating, or replication competent. Chimeric viral vectors which exploit advantageous elements of each of the parent vector properties (See, e.g., Feng et al. (1997) NATURE BIOTECHNOLOGY 15:866-870) may also be useful in the practice of the present invention. Although it is generally favored to employ a virus from the species to be treated, in some instances it may be advantageous to use vectors derived from different species that possess favorable pathogenic features. For example, equine herpes virus vectors for human gene therapy are described in PCT Publication No. WO 98/27216. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in PCT Publication No. WO 97/06826.

In certain embodiments, the viral vector is an oncolytic virus, e.g., a virus that exhibits tumor-selective replication and/or viral mediated lysis. In certain embodiments, the oncolytic virus allows for selective expression of a disclosed fusion protein, e.g., the virus permits expression of the fusion protein in neoplastic cells, but attenuates expression in normal cells. In certain embodiments, the expression of the fusion protein in a non-hyperproliferative cell is about 90%, about 80%, about 70%, about 60%, about 50%, about 40%, about 30%, about 20%, about 10%, or about 5% of the expression of in a hyperproliferative cell. In certain embodiments, the virus exhibits no detectable expression of the fusion protein in a non-hyperproliferative cell. Fusion protein expression may be determined by any appropriate method known in the art, e.g., Western blot or ELISA. The hyperproliferative cell may be a cancer cell, e.g., a carcinoma, sarcoma, leukemia, lymphoma, prostate cancer, lung cancer, gastrointestinal tract cancer, colorectal cancer, pancreatic cancer, breast cancer, ovarian cancer, cervical cancer, stomach cancer, thyroid cancer, mesothelioma, liver cancer, kidney cancer, skin cancer, head and neck cancer, or brain cancer cell.

Preferably, the viral vector is an adenovirus. Adenoviruses are medium-sized (90-100 nm), non-enveloped (naked), icosahedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera. In particular, human adenoviruses includes the A-F subgenera as well as the individual serotypes thereof, the individual serotypes and A-F subgenera including but not limited to human adenovirus types 1, 2, 3, 4, 4a, 5, 6, 7, 8, 9, 10, 11 (Ad11a and Ad11p), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Preferred are vectors derived from human adenovirus types 2 and 5. Unless stated otherwise, all adenovirus type 5 nucleotide numbers are relative to the NCBI reference sequence AC_000008.1, which is depicted herein in SEQ ID NO: 52.

The adenovirus replication cycle has two phases: an early phase, during which 4 transcription units (E1, E2, E3, and E4) are expressed, and a late phase which occurs after the onset of viral DNA synthesis, and during which late transcripts are expressed primarily from the major late promoter (MLP). The late messages encode most of the virus's structural proteins. The gene products of E1, E2 and E4 are responsible for transcriptional activation, cell transformation, viral DNA replication, as well as other viral functions, and are necessary for viral growth.

The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

In certain embodiments, the virus has one or more modifications to a regulatory sequence or promoter. A modification to a regulatory sequence or promoter comprises a deletion, substitution, or addition of one or more nucleotides compared to the wild-type sequence of the regulatory sequence or promoter.

In certain embodiments, the modification of a regulatory sequence or promoter comprises a modification of a sequence of a transcription factor binding site to reduce affinity for the transcription factor, for example, by deleting a portion thereof, or by inserting a single point mutation into the binding site. In certain embodiments, the additional modified regulatory sequence enhances expression in neoplastic cells, but attenuates expression in normal cells.

In certain embodiments, the modified regulatory sequence is operably linked to a sequence encoding a protein. In certain embodiments, at least one of the adenoviral E1a and E1b genes (coding regions) is operably linked to a modified regulatory sequence. In certain embodiments, the E1a gene is operably linked to the modified regulatory sequence.

The E1a regulatory sequence contains five binding sites for the transcription factor Pea3, designated Pea3 I, Pea3 II, Pea3 III, Pea3 IV, and Pea3 V, where Pea3 I is the Pea3 binding site most proximal to the E1a start site, and Pea3 V is most distal. The E1a regulatory sequence also contains binding sites for the transcription factor E2F, hereby designated E2F I and E2F II, where E2F I is the E2F binding site most proximal to the E1a start site, and E2F II is more distal. From the E1a start site, the binding sites are arranged: Pea3 I, E2F I, Pea3 II, E2F II, Pea3 III, Pea3 IV, and Pea3 V.

In certain embodiments, at least one of these seven binding sites, or a functional portion thereof, is deleted. A “functional portion” is a portion of the binding site that, when deleted, decreases or even eliminates the functionality, e.g. binding affinity, of the binding site to its respective transcription factor (Pea3 or E2F) by, for example, at least 40%, 50%, 60%, 70%, 80%, 90%, 95% or 100% relative to the complete sequence. In certain embodiments, one or more entire binding sites are deleted. In certain embodiments, a functional portion of one or more binding sites is deleted. A “deleted binding site” encompasses both the deletion of an entire binding site and the deletion of a functional portion thereof. When two or more binding sites are deleted, any combination of entire binding site deletion and functional portion deletion may be used.

In certain embodiments, at least one Pea3 binding site, or a functional portion thereof, is deleted. The deleted Pea3 binding site can be Pea3 I, Pea3 II, Pea3 III, Pea3 IV, and/or Pea3 V. In certain embodiments, the deleted Pea3 binding site is Pea3 II, Pea3 III, Pea3 IV, and/or Pea3 V. In certain embodiments, the deleted Pea3 binding site is Pea3 IV and/or Pea3 V. In certain embodiments, the deleted Pea3 binding site is Pea3 II and/or Pea3 III. In certain embodiments, the deleted Pea3 binding site is both Pea3 II and Pea3 III. In certain embodiments, the Pea3 I binding site, or a functional portion thereof, is retained.

In certain embodiments, at least one E2F binding site, or a functional portion thereof, is deleted. In certain embodiments, at least one E2F binding site, or a functional portion thereof, is retained. In certain embodiments, the retained E2F binding site is E2F I and/or E2F II. In certain embodiments, the retained E2F binding site is E2F II. In certain embodiments, the total deletion consists essentially of one or more of Pea3 II, Pea3 III, Pea3 IV, and/or Pea3 V, or functional portions thereof.

In certain embodiments, the virus has a deletion of a 50 base pair region located from −305 to −255 upstream of the E1a initiation site, e.g., corresponding to 195-244 of the Ad5 genome (SEQ ID NO: 52), hereafter referred to as the TAV-255 deletion. In certain embodiments, the TAV-255 deletion results in an E1a promoter that comprises the sequence GGTGTTTTGG (SEQ ID NO: 55).

The adenoviral E1b-19k gene functions primarily as an anti-apoptotic gene and is a homolog of the cellular anti-apoptotic gene, BCL-2. Since host cell death prior to maturation of the progeny viral particles would restrict viral replication, E1b-19k is expressed as part of the E1 cassette to prevent premature cell death thereby allowing the infection to proceed and yield mature virions. Accordingly, in certain embodiments, a recombinant virus is provided that includes an E1b-19K insertion site, e.g., the adenovirus has an exogenous nucleotide sequence encoding a disclosed fusion protein inserted into an E1b-19K insertion site.

In certain embodiments, the E1b-19K insertion site is located between the start site of E1b-19K (i.e., the nucleotide sequence encoding the start codon of E1b-19k, e.g., corresponding to nucleotides 1714-1716 of SEQ ID NO: 52) and the start site of E1b-55K (i.e., the nucleotide sequence encoding the start codon of E1b-55k, e.g., corresponding to nucleotides 2019-2021 of SEQ ID NO: 52). Throughout the description and claims, an insertion between two sites, for example, an insertion between (i) a start site of a first gene (e.g., E1b-19k) and a start site of a second gene, (e.g., E1b-55K), (ii) a start site of a first gene and a stop site of a second gene, (iii) a stop site of a first gene and start site of a second gene, or (iv) a stop site of first gene and a stop site of a second gene, is understood to mean that all or a portion of the nucleotides constituting a given start site or a stop site surrounding the insertion may be present or absent in the final virus. Similarly, an insertion between two nucleotides is understood to mean that the nucleotides surrounding the insertion may be present or absent in the final virus.

In certain embodiments, the E1b-19K insertion site is located between the start site of E1b-19K (i.e., the nucleotide sequence encoding the start codon of E1b-19k, e.g., corresponding to nucleotides 1714-1716 of SEQ ID NO: 52) and the stop site of E1b-19K (i.e., the nucleotide sequence encoding the stop codon of E1b-19k, e.g., corresponding to nucleotides 2242-2244 of SEQ ID NO: 52). In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 203 nucleotides adjacent the start site of E1b-19K. In certain embodiments, the E1b-19K insertion site comprises a deletion corresponding to nucleotides 1714-1916 of the Ad5 genome (SEQ ID NO: 52), or the exogenous nucleotide sequence is inserted between nucleotides corresponding to 1714 and 1916 of the Ad5 genome (SEQ ID NO: 52). In certain embodiments, the exogenous nucleotide sequence is inserted between CTGACCTC and TCACCAGG, e.g., the recombinant adenovirus comprises, in a 5′ to 3′ orientation, CTGACCTC, the exogenous nucleotide sequence, and TCACCAGG. CTGACCTC and TCACCAGG define unique boundary sequences for the E1b-19K insertion site within the Ad5 genome (SEQ ID NO: 52). Throughout the description and claims, a deletion adjacent to a site, for example, a deletion adjacent to a start site of a gene or a deletion adjacent to a stop site of a gene, is understood to mean that the deletion may include a deletion of all, a portion, or none of the nucleotides constituting a given start site or a stop site.

In certain embodiments the recombinant adenovirus comprises an E3 deletion. In certain embodiments, the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides.

In certain embodiments, the E3 deletion comprises a deletion located between the stop site of pVIII (i.e., the nucleotide sequence encoding the stop codon of pVIII, e.g., corresponding to nucleotides 27855-27857 of SEQ ID NO: 52) and the start site of Fiber (i.e., the nucleotide sequence encoding the start codon of Fiber, e.g., corresponding to nucleotides 31042-31044 of SEQ ID NO: 52). In certain embodiments, the E3 deletion comprises a deletion located between the stop site of E3-10.5K (i.e., the nucleotide sequence encoding the stop codon of E3-10.5K, e.g., corresponding to nucleotides 29770-29772 of SEQ ID NO: 52) and the stop site of E3-14.7K (i.e., the nucleotide sequence encoding the stop codon of E3-14.7K, e.g., corresponding to nucleotides 30837-30839 of SEQ ID NO: 52). In certain embodiments, the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent to the stop site of E3-10.5K. In certain embodiments, the E3 deletion comprises a deletion of about 1050 nucleotides adjacent to the stop site of E3-10.5K (i.e., the nucleotide sequence encoding the stop codon of E3-10.5K, e.g., corresponding to nucleotides 29770-29772 of SEQ ID NO: 52), e.g., the E3 deletion comprises a deletion of 1064 nucleotides adjacent to the stop site of E3-10.5K. In certain embodiments, the E3 deletion comprises a deletion corresponding to the Ad5 dl309 E3 deletion. In certain embodiments, the E3 deletion comprises a deletion corresponding to nucleotides 29773-30836 of the Ad5 genome (SEQ ID NO: 52).

In certain embodiments, the E3 deletion comprises a deletion located between the stop site of E3-gp19K (i.e., the nucleotide sequence encoding the stop codon of E3-gp19K, e.g., corresponding to nucleotides 29215-29217 of SEQ ID NO: 52) and the stop site of E3-14.7K (i.e., the nucleotide sequence encoding the stop codon of E3-14.7K, e.g., corresponding to nucleotides 30837-30839 of SEQ ID NO: 52). In certain embodiments, the E3 deletion comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 deletion comprises a deletion of about 1600 nucleotides adjacent the stop site of E3-gp19K. e.g., the E3 insertion site comprises a deletion of 1622 nucleotides adjacent the stop site of E3-gp19K. In certain embodiments, the E3 deletion comprises a deletion corresponding to nucleotides 29218-30839 of the Ad5 genome (SEQ ID NO: 52).

In certain embodiments, the recombinant adenovirus comprises an E3 insertion site, e.g., the adenovirus has an exogenous nucleotide sequence encoding a disclosed fusion protein inserted into the E3 deletion. For example, in certain embodiments, an exogenous nucleotide sequence is inserted between nucleotides corresponding to 29773 and 30836 of the Ad5 genome (SEQ ID NO: 52). In certain embodiments, the exogenous nucleotide sequence is inserted between CAGTATGA and TAATAAAAAA (SEQ ID NO: 57), e.g., the recombinant adenovirus comprises, in a 5′ to 3′ orientation, CAGTATGA, the exogenous nucleotide sequence, and TAATAAAAAA (SEQ ID NO: 57). CAGTATGA and TAATAAAAAA (SEQ ID NO: 57) define unique boundary sequences for an E3 insertion site within the Ad5 genome (SEQ ID NO: 52).

In certain embodiments, the exogenous nucleotide sequence is inserted between nucleotides corresponding to 29218 and 30839 of the Ad5 genome (SEQ ID NO: 52). In certain embodiments, the exogenous nucleotide sequence is inserted between TGCCTTAA and TAAAAAAAAAT (SEQ ID NO: 59), e.g., the recombinant adenovirus comprises, in a 5′ to 3′ orientation, TGCCTTAA, the exogenous nucleotide sequence, and TAAAAAAAAAT (SEQ ID NO: 59). TGCCTTAA and TAAAAAAAAAT (SEQ ID NO: 59) define unique boundary sequences for an E3 insertion site within the Ad5 genome (SEQ ID NO: 52).

Additional exemplary adenovirus vectors useful in the practice of this aspect of the invention are described in U.S. Pat. No. 9,073,980.

### IV. Fusion Protein Modifications

When used as a therapeutic, a fusion protein may be optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into a disclosed fusion protein by DNA shuffling, chain shuffling, CDR shuffling, random mutagenesis and/or site-specific mutagenesis.

Generally, an optimized fusion protein has at least the same, or substantially the same (e.g., at least 85%, 90%, 95%, 96%, 97%, 98%, or 99%) affinity for a ligand as the non-optimized (or parental) fusion protein from which it was derived. Preferably, an optimized fusion protein has a higher affinity for a ligand when compared to a parental fusion protein.

Fusion proteins (e.g., parental and optimized variants) can be engineered to contain certain constant (i.e., Fc) regions with a specified effector function (e.g., antibody-dependent cellular cytotoxicity (ADCC)). Human constant regions are known in the art.

Furthermore, if the fusion protein is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

### V. Methods of Treatment

The foregoing fusion proteins or expression vectors can be used to treat various medical indications. In certain embodiments, the foregoing fusion proteins or expression vectors can be used to treat medical indications that are mediated by a cytokine, for example TGFβ. For example, the fusion proteins and expression vectors can be used to treat various cancers or inflammatory diseases.

As used herein, “treat,” “treating” and “treatment” mean the treatment of a disease in a subject, e.g., in a mammal, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

In certain embodiments, the fusion proteins and expression vectors disclosed herein can be used to treat various cancers. The cancer cells are exposed to a therapeutically effective amount of the fusion protein or expression vector so as to inhibit or reduce proliferation of the cancer cells. In certain embodiments, administering a therapeutically effective amount of a fusion protein or expression vector to cancer cells reduces TGFβ in the cells by at least 30%, at least 40%, at least 50%, at least 60%, at least 70%, at least 80%, at least 90%, or at least 95%. TGFβ activity may be assayed by Western blot as described in Example 2. In some embodiments, a disclosed fusion protein or expression vector can be used to inhibit tumor growth in a subject (e.g., a human patient, also referred to as a human subject), which can be accomplished by administering an effective amount of the fusion protein or expression vector to the subject. In certain embodiments, administering an effective amount of a fusion protein or expression vector to a subject reduces tumor load in that subject by at least 30%, at least 40%, at least 50%, at least 60%, at least 70%, at least 80%, or at least 90%.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments, the cancer is selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, liver cancer, hepatocellular carcinoma, cholangiocarcinoma, brain and central nervous system cancer, thyroid cancer, parathyroid cancer (e.g., parathyroid carcinoma), endometrial cancer, neuroendocrine cancer, lymphoma (e.g., Hodgkin and non-Hodgkin), leukemia, merkel cell carcinoma, gastrointestinal stromal tumors, multiple myeloma, uterine cancer, a sarcoma, kidney cancer, ocular cancer, pancreatic cancer, and a germ cell cancer (e.g., ovarian germ cell cancer). In certain embodiments, the cancer may be selected from leukemia, breast cancer, lung cancer, pancreatic cancer, endometrial cancer, ovarian cancer, prostate cancer, cervical cancer, brain cancer, skin cancer, colorectal cancer, gastric cancer, head and neck cancer, and leukemia. In certain embodiments, the cancer is selected from leukemia, breast cancer, cervical cancer, colorectal cancer, lung cancer, pancreatic cancer, prostate cancer, gastric cancer, head and neck cancer, endometrial cancer and ovarian cancer.

In certain embodiments, a fusion protein or expression vector of the disclosure is administered to decrease levels of one or more cytokines in a subject in need thereof (e.g., a subject with an inflammatory condition). In certain embodiments, a disclosed fusion protein or expression vector can be used to treat an inflammatory condition in a subject (e.g., a human subject), which can be accomplished by administering an effective amount of the fusion protein or expression vector to the subject.

As used herein, an inflammatory condition is a disease or condition characterized, in whole or in part, by inflammation or an inflammatory response in the patient. Inflammatory conditions treatable using the fusion proteins or expression vectors of the invention may be characterized, for example, based on the primary tissue affected, the mechanism of action underlying the condition, or the portion of the immune system that is misregulated or overactive. In certain embodiments, examples of inflammatory conditions that may be treated include inflammation of the lungs (e.g., asthma, adult respiratory distress syndrome, bronchitis, pulmonary inflammation, pulmonary fibrosis, and cystic fibrosis), joints (e.g., rheumatoid arthritis, rheumatoid spondylitis, juvenile rheumatoid arthritis, osteoarthritis, gouty arthritis and other arthritic conditions), connective tissue, eyes (e.g., uveitis (including iritis), conjunctivitis, scleritis, and keratoconjunctivitis sicca), nose, bowel (e.g., Crohn's disease, ulcerative colitis, inflammatory bowel disease, inflammatory bowel syndrome, and distal proctitis), kidney (e.g., glomerulonephritis, interstitial nephritis, lupus nephritis, nephritis secondary to Wegener's disease, acute renal failure secondary to acute nephritis, Goodpasture's syndrome, post-obstructive syndrome and tubular ischemia), liver (e.g., hepatitis (arising from viral infection, autoimmune responses, drug treatments, toxins, environmental agents, or as a secondary consequence of a primary disorder), obesity, biliary atresia, primary biliary cirrhosis and primary sclerosing cholangitis), skin (e.g., psoriasis, eczema, and dermatitis, e. g., eczematous dermatitides, topic and seborrheic dermatitis, allergic or irritant contact dermatitis, eczema craquelee, photoallergic dermatitis, phototoxicdermatitis, phytophotodermatitis, radiation dermatitis, and stasis dermatitis), central nervous system (e.g., multiple sclerosis and neurodegenerative diseases such as Alzheimer's disease, Parkinson's disease or dementia associated with HIV infection), vascular system (e.g. coronary infarct damage, peripheral vascular disease, myocarditis, vasculitis, revascularization of stenosis, atherosclerosis, and vascular disease associated with Type II diabetes), endocrine system (e.g., autoimmune thyroiditis (Hashimoto's disease), Type I diabetes, inflammation in liver and adipose tissue associated with Type II diabetes, and acute and chronic inflammation of the adrenal cortex) heart, or adipose tissue. The disclosure contemplates that some inflammatory conditions involve inflammation in multiple tissues. Moreover, the disclosure contemplates that some inflammatory conditions may fall into multiple categories. In certain embodiments, the inflammatory condition is an autoimmune disease. Exemplary autoimmune diseases include, but are not limited to, rheumatoid arthritis, psoriasis (including plaque psoriasis), psoriatic arthritis, ankylosing spondylitis, ulcerative colitis, multiple sclerosis, lupus, alopecia, autoimmune pancreatitis, Celiac disease, Behcet's disease, Cushing syndrome, and Grave's disease. In certain embodiments, the inflammatory condition is a rheumatoid disorder. Exemplary rheumatoid disorders include, but are not limited to, rheumatoid arthritis, juvenile arthritis, bursitis, spondylitis, gout, scleroderma, Still's disease, and vasculitis. It is noted that certain categories of conditions overlap. For example, rheumatoid arthritis is an inflammatory rheumatoid disorder, an inflammatory joint disorder, and an autoimmune disorder.

The term “effective amount” as used herein refers to the amount of an active component (e.g., the amount of a fusion protein or expression vector of the present invention) sufficient to effect beneficial or desired results. An effective amount can be administered in one or more administrations, applications or dosages and is not intended to be limited to a particular formulation or administration route.

In certain embodiments, a therapeutically effective amount of a fusion protein is in the range of 0.1 mg/kg to 100 mg/kg, e.g., 1 mg/kg to 100 mg/kg, 1 mg/kg to 10 mg/kg, 1 mg/kg to 5 mg/kg, 10 mg/kg, 7.5 mg/kg, 5 mg/kg, or 2.5 mg/kg. In certain embodiments, a therapeutically effective amount of an expression vector, e.g., a recombinant virus, is in the range of 10² to 10¹⁵ plaque forming units (pfus), e.g., 10² to 10¹⁰, 10² to 10⁵, 10⁵ to 10¹⁵, 10⁵ to 10¹⁰, or 10¹⁰ to 10¹⁵ plaque forming units. The amount administered will depend on variables such as the type and extent of disease or indication to be treated, the overall health of the patient, the in vivo potency of the fusion protein or expression vector, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks. A preferred route of administration is parenteral, e.g., intravenous infusion. Formulation of fusion protein-or expression vector-based drugs is within ordinary skill in the art. In some embodiments, a fusion protein or expression vector is lyophilized, and then reconstituted in buffered saline, at the time of administration.

For therapeutic use, a fusion protein or expression vector preferably is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing fusion proteins or expression vectors disclosed herein can be presented in a dosage unit form and can be prepared by any suitable method. A pharmaceutical composition should be formulated to be compatible with its intended route of administration. Examples of routes of administration are intravenous (IV), intradermal, inhalation, intraocular, intranasal, transdermal, topical, transmucosal, and rectal administration.

A preferred route of administration for fusion proteins is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution. In certain embodiments, a delivery vehicle (e.g., a recombinant virus) and/or a therapeutic agent of the invention is administered in combination with a checkpoint inhibitor, e.g., an anti-CTLA-4 antibody, an anti-PD-1 antibody, or an anti-PD-L1 antibody. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies include, for example, atezolizumab (Tecentriq®, Genentech), duvalumab (AstraZeneca), MEDI4736, avelumab (Bavencio®, EMD Serono), and BMS 936559 (Bristol Myers Squibb Co.).

The term administered “in combination,” as used herein, is understood to mean that two (or more) different treatments are delivered to the subject during the course of the subject's affliction with the disorder, such that the effects of the treatments on the subject overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Throughout the description, where compositions, devices, and systems are described as having, including, or comprising specific components, or where processes and methods are described as having, including, or comprising specific steps, it is contemplated that, additionally, there are compositions, devices, and systems of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular virus, that virus can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1: TGFβR Fusion Protein Plasmid and Adenovirus Construction

This Example describes the production of plasmids and viral expression vectors that encode TGFβR fusion proteins.

To construct a nucleotide sequence encoding a mouse TGFβR-IgG1 fusion protein (mTGFβR-IgG1), plasmids pORF9-mIL10RA, PUNO1-mTGFBR2, and pFUSEss-CHIg-mG1 were purchased from Invivogen. The pUNO1-mTGFBR2 plasmid was cleaved with KasI and NheI to release a 1.7 kb fragment with the coding region of the mouse TGFβ type 2 receptor. The pORF9-mIL10RA plasmid was cleaved with KasI and NheI to release a 3 kb fragment containing the vector backbone. Those two fragments were ligated to generate the plasmid pORF9-TGFBR2.

The plasmid pORF9-TGFBR2 was amplified with primers flanking the KasI site 5′ of the coding region and either a primer corresponding to the 3′ end of the extracellular domain followed by an NheI site to produce only the extracellular domain, or a primer corresponding to the 3′ end of the extracellular domain followed by a portion of the mouse IgG1 (mIgG1) CH1 domain to produce the 5′ half of a fusion gene. The plasmid pFUSEss-CHIg-mG1 was amplified with primers corresponding to the 3′ end of the mIgG1 gene followed by a NheI site, and the 3′ end of the extracellular domain of the mTGFβR followed by a portion of the mIgG1 CH1 domain. Fusion genes were generated by combining these PCR products in a second round PCR reaction. PCR products were then cleaved with KasI and NheI and ligated into a pORF9 backbone cleaved with the same enzymes to generate pORF9 plasmids carrying either the extracellular domain or the mIgG1 fusion genes. The resulting nucleotide sequence encoded a fusion protein (mTGFBR-IgG, SEQ ID NO: 33) including residues 1-159 of the mTGFβR sequence (ending in TSSPD) immediately followed by residues 90-324 of the mIgG1 sequence, starting at the beginning of the final B strand of the second immunoglobulin fold (beginning with STKVD).

To construct nucleotide sequences encoding human TGFβR-IgG1 fusion proteins, plasmids carrying cDNA of human IgG1 (hIgG1, Accession BC072419 in pCMV-SPORT6) and human TGFβ receptor type 2 (Accession BC040499 in pBluescript®) were purchased from Thermo Scientific. PCR amplification using a 5′ primer carrying a SalI site, a 3′ primer carrying an XhoI site, and linking primers carrying a sequence from the 3′ end of hTGFβR and the 5′ end of hIgG1 was performed as described for the mouse genes.

Nucleotide sequences encoding a series of fusion proteins were generated. A first fusion protein, hTGFβR-IgG1-1 (SEQ ID NO: 22), included residues 1-159 of hTGFβR (ending in TSNPD), immediately followed by residues 88-330 of hIgG1, starting at the beginning of the final B strand of the second immunoglobulin fold (beginning at KPSNT). A second fusion protein, hTGFβR-IgG1-2 (SEQ ID NO: 62), included residues 1-159 of hTGFβR (ending in TSNPD), immediately followed by residues 90-330 of hIgG1 (beginning at SNTKV). A third fusion protein, hTGFβR-IgG1-3 (SEQ ID NO: 63), included residues 1-159 of hTGFβR (ending in TSNPD), immediately followed by residues 92-330 of hIgG1 (beginning at TKVDK). A fourth fusion protein, hTGFβR-IgG1-4, included residues 1-159 of hTGFβR (ending in TSNPD), immediately followed by residues 94-330 of hIgG1 (beginning at VDKRV). A fifth fusion protein, hTGFβR-Fc (SEQ ID NO: 48), included residues 1-159 of TGFβR (ending in TSNPD), immediately followed by residues 100-330 of hIgG1 (beginning at PKSCD). The fifth fusion protein was referred to as hTGFβR-Fc because it included only the Fc domain and hinge region of the immunoglobulin, in contrast to hTGFβR-IgG-1, hTGFβR-IgG-2, hTGFβR-IgG-3, and hTGFβR-IgG-4, which included from six to twelve additional amino acids from hIgG1. Details of the fusion proteins are shown in TABLE 3.

### TABLE 3

TABLE 3

| Fusion | hTGFβR | hIgG1 | hTGFβR-hIgG1 |
|---|---|---|---|
| Protein | Residues | Residues | Junction |
| hTGFβR-IgG1-1 | 1-159 | 88-330 | TSNPD-KPSNTK |
| | | | VDKRVEPKSCD |
| hTGFβR-IgG1-2 | 1-159 | 90-330 | TSNPD-SNTKVD |
| | | | KRVEPKSCD |
| hTGFβR-IgG1-3 | 1-159 | 92-330 | TSNPD-TKVDKR |
| | | | VEPKSCD |
| hTGFβR-IgG1-4 | 1-159 | 94-330 | TSNPD-VDKRVE |
| | | | PKSCD |
| hTGFβR-Fc | 1-159 | 100-330 | TSNPD-PKSCD |

Nucleotide sequences encoding the fusion proteins were cloned into plasmids for downstream applications as appropriate. For adenovirus construction, nucleotide sequences were cloned into a derivative of pXC1 (which carries the 5′ portion of the adenovirus genome), modified to carry a SalI site at the start site of the E1B-19k region and an XhoI site 200 base pairs 3′ of the SalI site. When indicated, pXC1 was further modified at the E1A promoter region to produce the plasmid pXC1-TAV-255, which renders E1A expression cancer-selective (as previously described in U.S. Pat. No. 9,073,980). PCR products were cloned into the pXC1 (or pXC1-TAV) backbone using InFusion (Clontech) according to the manufacturer's instructions.

Where indicated, the pXC1 plasmids were cotransfected with the plasmid pJM17 in HEK-293A cells to allow homologous recombination to rescue recombinant virus. Virus was collected and underwent two rounds of plaque purification and sequencing to confirm presence of the fusion gene and test for presence of the TAV-255 deletion as necessary. The virus carrying the mouse isoform was grown in 293 cells, and the virus carrying the human isoform was plaque purified and grown exclusively in A549 cells after the initial viral rescue in 293 cells. Virus to be used in animal experiments was purified using Fast-Trap adenovirus purification kits (Millipore), dialyzed into viral storage buffer (25 mM NaCl, 10 mM Tris pH 8, 5% glycerol), and stored at −80° until use. Details of the viruses tested are shown in TABLE 4.

### TABLE 4

TABLE 4

| | E1A | E1B-19k |
|---|---|---|
| Virus | Promoter | Modification |
| Wild-type | Wild-type | Wild-type |
| Ad-Control | Wild-type | Deleted |
| Ad-mTGFβR-IgG1 | Wild-type | Deleted and replaced |
| | | with mTGFβR-IgG1 |
| Ad-hTGFβR-IgG1-1 | TAV-255 | Deleted and replaced |
| | | with hTGFβR-IgG1-1 |

### Example 2: Inhibition of TGFβ Signaling

This Example describes a comparison between disclosed hTGFβR-IgG1 fusion proteins and conventional hTGFβR-IgG1 fusion proteins.

As described in Example 1, plasmids were generating encoding a series of human TGFB trap fusion proteins: hTGFβR-IgG1-1, hTGFβR-IgG1-2, hTGFβR-IgG1-3, hTGFβR-IgG1-4, and hTGFβR-Fc.

hTGFβR-Fc (SEQ ID NO: 48) contains amino acids Thr23 to Asp159 of the human TGFβ type II receptor and amino acids Pro 100 to Lys330 of human IgG1. This sequence is identical that used in a commercially available TGFβ trap fusion protein (R&D Systems).

In contrast to the conventional TGFB trap fusion protein, hTGFβR-IgG1-1 (SEQ ID NO: 22), hTGFβR-IgG-2 (SEQ ID NO: 62), hTGFβR-IgG-3 (SEQ ID NO: 63), and hTGFβR-IgG-4, contain twelve, ten, eight, or six amino acids, respectively, from the CH1 domain of IgG1 that serve as a flexible, non-immunogenic linker between the TGFβ type II receptor and the hinge and Fc region of the IgG1.

HEK-293 cells were transfected with pXC1 plasmids carrying hTGFβR-IgG1-1, hTGFβR-IgG1-2, hTGFβR-IgG1-3, hTGFβR-IgG1-4, or hTGFβR-Fc genes, or were kept as non-transfected controls, and were incubated for five days to allow protein expression and secretion into the media. The conditioned media was collected, TGFβ was added to the media at 500 pg/ml where indicated, and the media was then overlaid on fresh reporter cells and incubated for one hour. Free TGFβ will induce Smad2 phosphorylation in the reporter cells, however, if the TGFB trap fusion protein blocks TGFβ, then it will not lead to Smad2 phosphorylation. Protein extracts of the reporter cells were probed by Western blot for phosphorylated Smad2. B-actin was used as a loading control, or subsequently the blot was stripped and reprobed for total Smad2 and Smad3 to serve as a loading control.

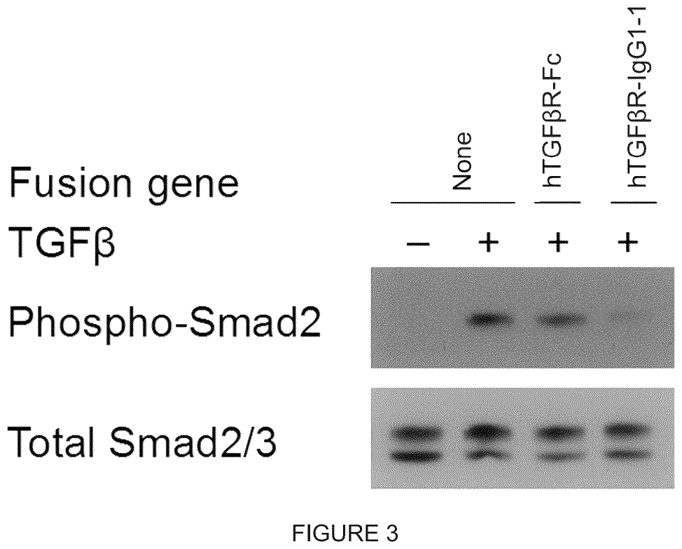

A comparison between hTGFβR-IgG1-1 and hTGFβR-Fc is shown in FIG. 3. As seen in FIG. 3, conditioned media from cells transfected with the conventional hTGFβR-Fc fusion gene has modest inhibition of TGFβ, while hTGFβR-IgG-1 more effectively blocked TGFβ signaling. Quantitation of the intensity of the Western blot shows that, compared to controls, hTGFβR-Fc resulted in a 21% reduction of TGFβ activity, and hTGFβR-IgG resulted in a 92% reduction of TGFβ activity.

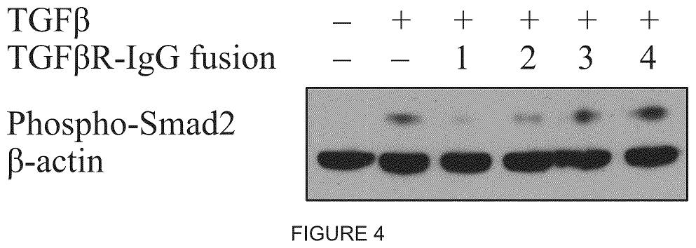

A comparison between hTGFβR-IgG1-1, hTGFβR-IgG1-2, hTGFβR-IgG1-3, and hTGFβR-IgG1-4 is shown in FIG. 4. As seen in FIG. 4, conditioned media from cells transfected with the hTGFβR-IgG1-1 and hTGFβR-IgG1-2 fusion genes effectively blocked TGFβ signaling.

Together, these results demonstrate that TGFβ activity was markedly reduced by disclosed hTGFβR-IgG1 fusion proteins, e.g., hTGFβR-IgG1-1 and hTGFβR-IgG1-2, compared to a conventional hTGFβR-IgG1 fusion protein, e.g., hTGFβR-Fc.

### Example 3: Inhibition of Tumor Growth

Experiments in mice were conducted using Ad-mTGFβR-IgG1, a virus carrying the mTGFβR-IgG1 fusion gene, in order to prevent the undesired induction of murine antibodies against the human TGFβR isoform. Ad-Control, a control virus in which the E1B-19k site used to carry the transgene was deleted, was also tested. The Ad-mTGFβR-IgG1 and Ad-Control viruses do not carry the 50 bp TAV-255 deletion, which serves as an attenuation mechanism to reduce viral replication in normal cells. Viruses were prepared as described in Example 1, and the key features of the viruses are shown schematically in TABLE 4 above.

Many mouse cells can be infected by human adenovirus with some degree of viral gene expression, but most mouse cell lines are not permissive for human adenovirus type 5 replication. ADS-12 is a mouse lung cancer cell line that was recently described as the first (and currently only) identified mouse cancer cell line that supports replication of human adenovirus at levels comparable to human cells, and was therefore chosen as a model system (Zhang et al. (2015) CANCER GENE THER. 22(1):17-22).

Mice carrying subcutaneous ADS-12 tumors were treated with intratumoral injections given every four days for three total doses of vehicle, Ad-Control, or Ad-mTGFβR-IgG1 at 10⁹ PFU/dose.

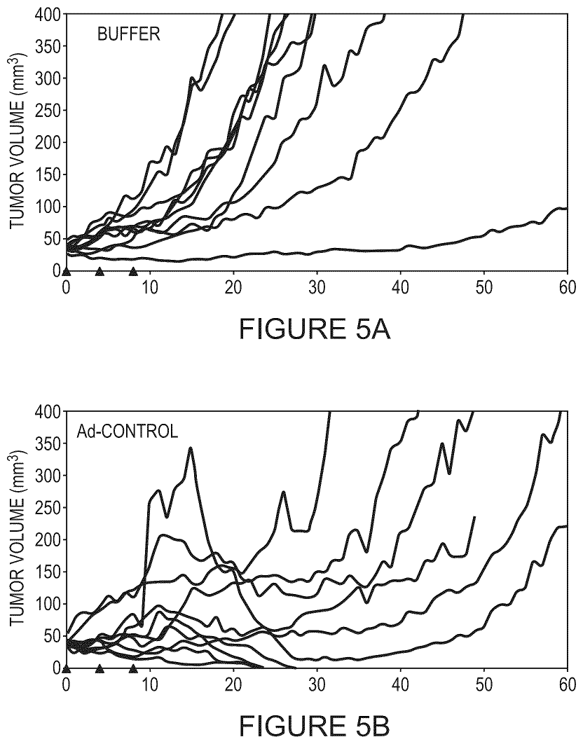

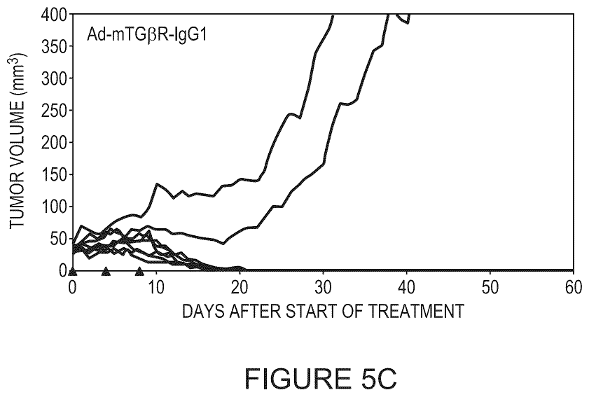

As shown in FIGS. 5A-5C, all tumors treated with intratumoral injections of buffer alone progressed. Four out of ten tumors treated with the “unarmed” Ad-Control virus completely regressed, indicative of oncolytic activity in the absence of tumor-specific TGFβ trap transgene expression. By contrast, eight out of ten tumors treated with Ad-mTGFβR-IgG1 completely regressed, demonstrating improved tumor kill with the transgene.

In summary, an oncolytic virus expressing a novel TGFβ trap disclosed herein showed significantly enhanced anti-tumor effects.

### Example 4: Inhibition of TGFβ Signaling in Cancer Cell Lines

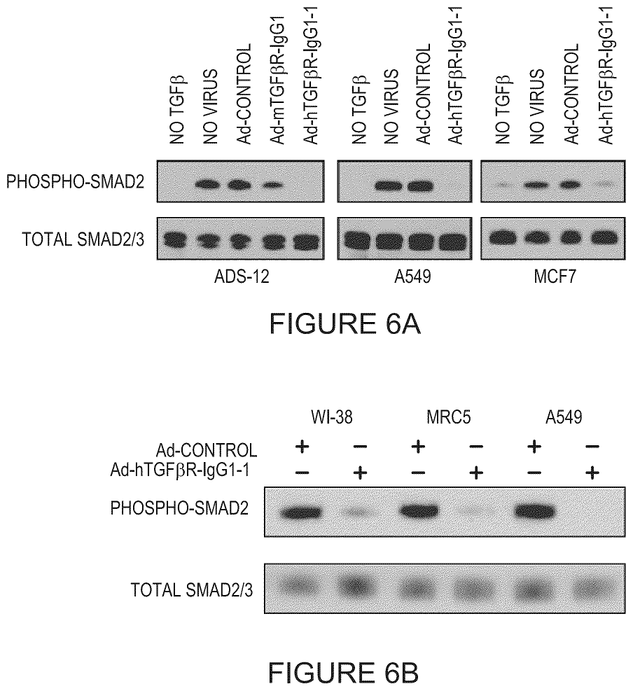

Assays on TGFβ inhibition were carried out in human cell lines using the Ad-hTGFβR-IgG1-1, Ad-mTGFβR-IgG1, and Ad-Control viruses. The viruses were prepared as described in Example 1 and the key features of the viruses are shown schematically in TABLE 4 above. Effects of virus were tested in normal (WI-38 and MRC5) and cancerous (ADS-12, A549, and MCF7) cells. Conditioned media from cells infected with the indicated virus was overlaid on fresh reporter cells and TGFβ added as described in Example 2. As seen in FIGS. 6A-6B, TGFβ induction of Smad2 phosphorylation was diminished in conditioned media from all cell lines infected with Ad-hTGFβR-IgG1-1. In summary, Ad-hTGFβR-IgG1-1 induced robust blockade of TGFβ in cancerous cells and even blunted TGFβ activity in infected normal cells.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

1-93. (canceled)

94. A method of inhibiting tumor growth in a subject in need thereof, the method comprising administering to the subject an effective amount of an expression vector to inhibit growth of the tumor,
wherein the expression vector comprises a nucleic acid comprising a nucleotide sequence encoding a fusion protein comprising, in an N- to C-terminal orientation:
(i) a soluble portion of an extracellular domain of a human TGFβ type II receptor wherein the soluble portion of the extracellular domain of the human TGFβ type II receptor has at least 90% identity to amino acid residues 23-159 of SEQ ID NO: 12 and binds to TGFβ;
(ii) an amino acid linker;
(iii) an immunoglobulin (Ig) hinge region; and
(iv) an immunoglobulin (Ig) Fc domain,
wherein the linker consists of 10 to 40 amino acid residues obtained from a C-terminal portion of a human immunoglobulin (Ig) CH1 domain and the linker comprises an amino acid sequence having at least 90% sequence identity to SEQ ID NO: 1, SEQ ID NO: 2, SEQ ID NO: 3, SEQ ID NO: 5, SEQ ID NO: 7, SEQ ID NO: 8, SEQ ID NO: 9, SEQ ID NO: 60, or SEQ ID NO: 61.

95. A method of treating cancer in a subject in need thereof, the method comprising administering an effective amount of an expression vector to the subject to treat the cancer,
wherein the expression vector comprises a nucleic acid comprising a nucleotide sequence encoding a fusion protein comprising, in an N- to C-terminal orientation:
(i) a soluble portion of an extracellular domain of a human TGFβ type II receptor wherein the soluble portion of the extracellular domain of the human TGFβ type II receptor has at least 90% identity to amino acid residues 23-159 of SEQ ID NO: 12 and binds to TGFβ;
(ii) an amino acid linker;
(iii) an immunoglobulin (Ig) hinge region; and
(iv) an immunoglobulin (Ig) Fc domain,
wherein the linker consists of 10 to 40 amino acid residues obtained from a C-terminal portion of a human immunoglobulin (Ig) CH1 domain and the linker comprises an amino acid sequence having at least 90% sequence identity to SEQ ID NO: 1, SEQ ID NO: 2, SEQ ID NO: 3, SEQ ID NO: 5, SEQ ID NO: 7, SEQ ID NO: 8, SEQ ID NO: 9, SEQ ID NO: 60, or SEQ ID NO: 61.

96. A method of reducing TGFβ activity in a cell of a subject, the method comprising exposing the cell to an effective amount of an expression vector to reduce TGFβ activity,
wherein the expression vector comprises a nucleic acid comprising a nucleotide sequence encoding a fusion protein comprising, in an N- to C-terminal orientation:
(i) a soluble portion of an extracellular domain of a human TGFβ type II receptor wherein the soluble portion of the extracellular domain of the human TGFβ type II receptor has at least 90% identity to amino acid residues 23-159 of SEQ ID NO: 12 and binds to TGFβ;
(ii) an amino acid linker;
(iii) an immunoglobulin (Ig) hinge region; and
(iv) an immunoglobulin (Ig) Fc domain,
wherein the linker consists of 10 to 40 amino acid residues obtained from a C-terminal portion of a human immunoglobulin (Ig) CH1 domain and the linker comprises an amino acid sequence having at least 90% sequence identity to SEQ ID NO: 1, SEQ ID NO: 2, SEQ ID NO: 3, SEQ ID NO: 5, SEQ ID NO: 7, SEQ ID NO: 8, SEQ ID NO: 9, SEQ ID NO: 60, or SEQ ID NO: 61.

97-98. (canceled)

99. The method of claim 95, wherein the cancer is selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, liver cancer, hepatocellular carcinoma, cholangiocarcinoma, brain and central nervous system cancer, thyroid cancer, parathyroid cancer, endometrial cancer, neuroendocrine cancer, lymphoma, leukemia, merkel cell carcinoma, gastrointestinal stromal tumors, multiple myeloma, uterine cancer, a sarcoma, kidney cancer, ocular cancer, pancreatic cancer, and a germ cell cancer.

100. The method of claim 95, wherein the cancer is selected from breast cancer, lung cancer, pancreatic cancer, endometrial cancer, ovarian cancer, prostate cancer, cervical cancer, brain cancer, skin cancer, colorectal cancer, gastric cancer, head and neck cancer, and leukemia.

101. The method of claim 100, wherein the cancer is selected from skin cancer, head and neck cancer, and lung cancer.

102. The method of claim 95, wherein the expression vector is administered in combination with one or more therapies selected from surgery, radiation, chemotherapy, immunotherapy, hormone therapy, and virotherapy.

103-107. (canceled)

108. The method of claim 95, wherein the cancer is a solid tumor.

109. The method of claim 108, wherein the expression vector is administered by intratumoral injection.

110. The method of claim 95, wherein the expression vector is an oncolytic virus.

111. The method of claim 110, wherein the oncolytic virus is an adenovirus and:
a) the nucleotide sequence encoding the fusion protein is inserted into an E1b-19K insertion site located between the start site of E1b-19K and the start site of E1b-55K and/or
b) the E1b-19K insertion site comprises a deletion corresponding to nucleotides 1714-1916 of the Ad5 genome (SEQ ID NO: 52); and/or
c) the nucleotide sequence encoding the fusion protein is inserted between nucleotides corresponding to 1714 and 1916 of the Ad5 genome (SEQ ID NO: 52); and/or
d) the nucleotide sequence encoding the fusion protein is inserted between CTGACCTC (SEQ ID NO: 53) and TCACCAGG (SEQ ID NO: 54); and/or
e) the adenovirus comprises, in a 5′ to 3′ orientation, CTGACCTC (SEQ ID NO: 53), the nucleotide sequence encoding the fusion protein, and TCACCAGG (SEQ ID NO: 54); and/or
f) the adenovirus comprises a deletion of a Pea3 binding site, or a functional fragment thereof.

112. The method of claim 110, wherein the expression vector selectively expresses the fusion protein in a hyperproliferative cell as a TGFβ binding protein.

113. The method of claim 95, wherein the Ig Fc domain and hinge region comprise an amino acid sequence having at least 90% sequence identity to SEQ ID NO: 13, SEQ ID NO: 14, SEQ ID NO: 15, SEQ ID NO: 16, SEQ ID NO: 17, SEQ ID NO: 18, SEQ ID NO:
19, SEQ ID NO: 20 or SEQ ID NO: 21.

114. The method of claim 95, wherein the fusion protein comprises the amino acid sequence of SEQ ID NO: 22.

115. The method of claim 95, wherein the fusion protein comprises the amino acid sequence of SEQ ID NO: 62.

116. The method of claim 95, wherein the fusion protein comprises the amino acid sequence of SEQ ID NO: 63.

117. The method of claim 102, wherein the expression vector is administered in combination with immunotherapy.

118. The method of claim 117, wherein the immunotherapy comprises a checkpoint inhibitor.

119. The method of claim 96, wherein the subject has an inflammatory condition.

120. The method of claim 96, wherein the subject has an infection.


SEQUENCE LISTING (94 sequences)

SEQ ID NO: 1 - protein, 12 aa, Homo sapiens
KPSNTKVDKKVE

SEQ ID NO: 2 - protein, 12 aa, Homo sapiens
KPSNTKVDKTVE

SEQ ID NO: 3 - protein, 12 aa, Homo sapiens
KPSNTKVDKRVE

SEQ ID NO: 4 - protein, 12 aa, Homo sapiens
KPSNTKVDKRVE

SEQ ID NO: 5 - protein, 12 aa, Homo sapiens
VKHYTNPSQDVT

SEQ ID NO: 6 - protein, 12 aa, Homo sapiens
VKHYTNPSQDVT

SEQ ID NO: 7 - protein, 10 aa, Homo sapiens
TASKSKKEIF

SEQ ID NO: 8 - protein, 14 aa, Homo sapiens
TPSSTDWVDNKTFS

SEQ ID NO: 9 - protein, 13 aa, Homo sapiens
VQHPNGNKEKNVP

SEQ ID NO: 10 - protein, 13 aa, Homo sapiens
VQHPNGNKEKNVP

SEQ ID NO: 11 - protein, 12 aa, Homo sapiens
REKQTDEIKDTR

SEQ ID NO: 12 - protein, 159 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 13 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 227 aa, Homo sapiens
RKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 15 - protein, 278 aa, Homo sapiens
LKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 16 - protein, 228 aa, Homo sapiens
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 255 aa, Homo sapiens
VPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 18 - protein, 242 aa, Homo sapiens
VPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 19 - protein, 286 aa, Homo sapiens
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 20 - protein, 325 aa, Homo sapiens
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 21 - protein, 351 aa, Homo sapiens
LPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 22 - protein, 402 aa, synthetic construct
  REGION 1..402: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 398 aa, synthetic construct
  REGION 1..398: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 24 - protein, 449 aa, synthetic construct
  REGION 1..449: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 25 - protein, 399 aa, synthetic construct
  REGION 1..399: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 426 aa, synthetic construct
  REGION 1..426: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 27 - protein, 413 aa, synthetic construct
  REGION 1..413: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 28 - protein, 455 aa, synthetic construct
  REGION 1..455: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 29 - protein, 498 aa, synthetic construct
  REGION 1..498: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 30 - protein, 523 aa, synthetic construct
  REGION 1..523: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 31 - protein, 402 aa, synthetic construct
  REGION 1..402: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDYVPKEFNAETFTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 402 aa, synthetic construct
  REGION 1..402: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDREKQTDEIKDTRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 394 aa, synthetic construct
  REGION 1..394: note = Synthetic Construct
MGRGLLRGLWPLHIVLWTRIASTIPPHVPKSVNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 159 aa, Mus musculus
MGRGLLRGLWPLHIVLWTRIASTIPPHVPKSVNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPD

SEQ ID NO: 35 - protein, 12 aa, Mus musculus
STKVDKKIVPRD

SEQ ID NO: 36 - protein, 223 aa, Mus musculus
CGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 37 - DNA, 1209 bp, synthetic construct
  misc_feature 1..1209: note = Synthetic Construct
  source 1..1209: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - DNA, 1194 bp, synthetic construct
  misc_feature 1..1194: note = Synthetic Construct
  source 1..1194: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 39 - DNA, 1347 bp, synthetic construct
  misc_feature 1..1347: note = Synthetic Construct
  source 1..1347: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaagcccagcaacaccaaggtggacaagagagttgagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 40 - DNA, 1197 bp, synthetic construct
  misc_feature 1..1197: note = Synthetic Construct
  source 1..1197: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 41 - DNA, 1278 bp, synthetic construct
  misc_feature 1..1278: note = Synthetic Construct
  source 1..1278: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacgtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgagcgtgacctctgtggctgctacagcgtgtccagtgtcctgccgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctac

SEQ ID NO: 42 - DNA, 1239 bp, synthetic construct
  misc_feature 1..1239: note = Synthetic Construct
  source 1..1239: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacgtgaagcactacacgaatcccagccaggatgtgactgtgccctgcccagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcatggcgggtaaacccacccatgtcaatgtgtctgttgtcatggcggaggtggacggcacctgctac

SEQ ID NO: 43 - DNA, 1365 bp, synthetic construct
  misc_feature 1..1365: note = Synthetic Construct
  source 1..1365: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccgccagcaagagtaagaaggagatcttccgctggccagagtctccaaaggcacaggcctcctcagtgcccactgcacaaccccaagcagagggcagcctcgccaaggcaaccacagccccagccaccacccgtaacacaggaagaggaggagaagagaagaagaaggagaaggagaaagaggaacaagaagagagagagacaaagacaccagagtgtccgagccacacccagcctcttggcgtctacctgctaacccctgcagtgcaggacctgtggctccgggacaaagccaccttcacctgcttcgtggtgggcagtgacctgaaggatgctcacctgacctgggaggtggccgggaaggtccccacagggggcgtggaggaagggctgctggagcggcacagcaacggctcccagagccagcacagccgtctgaccctgcccaggtccttgtggaacgcggggacctccgtcacctgcacactgaaccatcccagcctcccaccccagaggttgatggcgctgagagaacccgctgcgcaggcacccgtcaagctttccctgaacctgctggcctcgtctgaccctcccgaggcggcctcgtggctcctgtgtgaggtgtctggcttctcgccccccaacatcctcctgatgtggctggaggaccagcgtgaggtgaacacttctgggtttgcccccgcacgcccccctccacagcccgggagcaccacgttctgggcctggagtgtgctgcgtgtcccagccccgcccagccctcagccagccacctacacgtgtgtggtcagccacgaggactcccggactctgctcaacgccagccggagcctagaagtcagctatgtaacagaccatggccccatgaaa

SEQ ID NO: 44 - DNA, 1494 bp, synthetic construct
  misc_feature 1..1494: note = Synthetic Construct
  source 1..1494: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacactccatcgtccacagactgggtcgacaacaaaaccttcagcgtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaa

SEQ ID NO: 45 - DNA, 1569 bp, synthetic construct
  misc_feature 1..1569: note = Synthetic Construct
  source 1..1569: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacgtccagcaccccaacggcaacaaagaaaagaacgtgcctcttccagtgattgctgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccggtaaacccaccctgtacaacgtgtccctggtcatgtccgacacagctggcacctgctac

SEQ ID NO: 46 - DNA, 1206 bp, synthetic construct
  misc_feature 1..1206: note = Synthetic Construct
  source 1..1206: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgactacgttcccaaagagtttaatgctgaaacattcacccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 47 - DNA, 1206 bp, synthetic construct
  misc_feature 1..1206: note = Synthetic Construct
  source 1..1206: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacagagaaaaacagactgatgaaatcaaggatactaggcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 390 aa, synthetic construct
  REGION 1..390: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 15 aa, synthetic construct
  REGION 1..15: note = Synthetic Construct
GGGGSGGGGSGGGGS

SEQ ID NO: 50 - skipped

SEQ ID NO: 51 - protein, 4 aa, synthetic construct
  REGION 1..4: note = Furin cleavage site
RAKR

SEQ ID NO: 52 - DNA, 35938 bp, Adenovirus type 5
  source 1..35938: mol_type = unassigned DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 53-54 - skipped (2 entries)

SEQ ID NO: 55 - DNA, 10 bp, Adenovirus type 5
  source 1..10: mol_type = unassigned DNA
ggtgttttgg

SEQ ID NO: 56 - skipped

SEQ ID NO: 57 - DNA, 10 bp, Adenovirus type 5
  source 1..10: mol_type = unassigned DNA
taataaaaaa

SEQ ID NO: 58 - skipped

SEQ ID NO: 59 - DNA, 11 bp, Adenovirus type 5
  source 1..11: mol_type = unassigned DNA
taaaaaaaaat

SEQ ID NO: 60 - protein, 10 aa, Homo sapiens
SNTKVDKKVE

SEQ ID NO: 61 - protein, 8 aa, Homo sapiens
TKVDKRVE

SEQ ID NO: 62 - protein, 400 aa, synthetic construct
  REGION 1..400: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 398 aa, synthetic construct
  REGION 1..398: note = Synthetic Construct
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 93 aa, Homo sapiens
PKVFPLSLCSTQPDGNVVIACLVQGFFPQEPLSVIWSESGQGVTARNFPPSQDASGDLYTTSSQLTLPATQCLAGKSVTCHVKHYTNPSQDVT

SEQ ID NO: 65 - protein, 93 aa, Homo sapiens
PKVFPLSLDSTPQDGNVVIACLVQGFFPQEPLSVTWSESGQNVTARNFPPSQDASGDLYTTSSQLTLPATQCPDGKSVTCHVKHYTNPSQDVT

SEQ ID NO: 66 - protein, 93 aa, Homo sapiens
PDVFPIISGCRHPKDNSPVVLACLITGYHPTSVTVTWYMGTQSQPQRTFPEIQRRDSYYMTSSQLSTPLQQWRQGEYKCVVQHTASKSEKEIF

SEQ ID NO: 67 - protein, 98 aa, Homo sapiens
PSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFS

SEQ ID NO: 68 - protein, 94 aa, Homo sapiens
PSVFPLAPSSKSTSGGTAALGCLVKDYFPEPVTVSWNSGALTSGVHTFPAVLQSSGLYSLSSVVTVPSSSLGTQTYICNVNHKPSNTKVDKKVE

SEQ ID NO: 69 - protein, 94 aa, Homo sapiens
PSVFPLAPCSRSTSESTAALGCLVKDYFPEPVTVSWNSGALTSGVHTFPAVLQSSGLYSLSSVVTVPSSNFGTQTYTCNVDHKPSNTKVDKTVE

SEQ ID NO: 70 - protein, 94 aa, Homo sapiens
PSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVE

SEQ ID NO: 71 - protein, 94 aa, Homo sapiens
PSVFPLAPCSRSTSESTAALGCLVKDYFPEPVTVSWNSGALTSGVHTFPAVLQSSGLYSLSSVVTVPSSSLGTKTYTCNVDHKPSNTKVDKRVE

SEQ ID NO: 72 - protein, 97 aa, Homo sapiens
PTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVP

SEQ ID NO: 73 - protein, 95 aa, Homo sapiens
PRLSLHRPALEDLLLGSEANLTCTLTGRDASGVTFTWTPSSGKSAVQGPPERDLCGCYSVSSVLPGCAEPWNHGKTFTCTAAYPESKTPLTATLS

SEQ ID NO: 74 - protein, 95 aa, Homo sapiens
PRLSLHRPALEDLLLGSEANLTCTLTGRDASGATFTWTPSSGKSAVQGPPERDLCGCYSVSSVLPGCAQPWNHGETFTCTAAHPELKTPLTANIT

SEQ ID NO: 75 - protein, 89 aa, Homo sapiens
PAVQDLWLRDKATFTCFVVGSDLKDAHLTWEVAGKVPTGGVEEGLLERHSNGSQSQHSRLTLPRSLWNAGTSVTCTLNHPSLPPQRLMA

SEQ ID NO: 76 - protein, 99 aa, Homo sapiens
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 77 - protein, 100 aa, Homo sapiens
PSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 78 - protein, 101 aa, Homo sapiens
PSVFLFPPKPKDTLMISRTPEVTCVVVDVSHEDPEVQFNWYVDGVEVIINAKTKPREEQFNSTFRVVSVLTVVHQDWLNGKEYKCKVSNKGLPAPIEKTIS

SEQ ID NO: 79 - protein, 100 aa, Homo sapiens
PSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTIS

SEQ ID NO: 80 - protein, 101 aa, Homo sapiens
PSVFLFPPKPKDTLMISRTPEVTCVVVDVSQEDPEVQFNWYVDGVEVIINAKTKPREEQFNSTYRVVSVLTVLHQDWLNGKEYKCKVSNKGLPSSIEKTIS

SEQ ID NO: 81 - protein, 101 aa, Homo sapiens
PKVSVFVPPRDGFEGNPRKSKLICQATGFSPRQIQVSWLREGKQVGSGVTTDQVQAEAKESGPTTYKVTSTLTIKESDWLGQSMFTCRVDHRGLTFQQNAS

SEQ ID NO: 82 - protein, 5 aa, synthetic construct
  REGION 1..5: note = Synthetic Construct
TSSPD

SEQ ID NO: 83 - protein, 5 aa, synthetic construct
  REGION 1..5: note = Synthetic Construct
STKVD

SEQ ID NO: 84 - protein, 5 aa, synthetic construct
  REGION 1..5: note = Synthetic Construct
TSNPD

SEQ ID NO: 85 - protein, 5 aa, synthetic construct
  REGION 1..5: note = Synthetic Construct
KPSNT

SEQ ID NO: 86 - protein, 5 aa, synthetic construct
  REGION 1..5: note = Synthetic Construct
SNTKV

SEQ ID NO: 87 - protein, 5 aa, synthetic construct
  REGION 1..5: note = Synthetic Construct
TKVDK

SEQ ID NO: 88 - protein, 5 aa, synthetic construct
  REGION 1..5: note = Synthetic Construct
VDKRV

SEQ ID NO: 89 - protein, 5 aa, synthetic construct
  REGION 1..5: note = Synthetic Construct
PKSCD

SEQ ID NO: 90 - protein, 22 aa, synthetic construct
  REGION 1..22: note = Synthetic Construct
TSNPDKPSNTKVDKRVEPKSCD

SEQ ID NO: 91 - protein, 20 aa, synthetic construct
  REGION 1..20: note = Synthetic Construct
TSNPDSNTKVDKRVEPKSCD

SEQ ID NO: 92 - protein, 18 aa, synthetic construct
  REGION 1..18: note = Synthetic Construct
TSNPDTKVDKRVEPKSCD

SEQ ID NO: 93 - protein, 16 aa, synthetic construct
  REGION 1..16: note = Synthetic Construct
TSNPDVDKRVEPKSCD

SEQ ID NO: 94 - protein, 10 aa, synthetic construct
  REGION 1..10: note = Synthetic Construct
TSNPDPKSCD